(19) United States
(12) Patent Application Publication
(10) Pub. No.: US 2024/0018072 A1
(43) Pub. Date: Jan. 18, 2024

(54) METHOD FOR REDUCING IMPURITIES IN TRIFLUOROIODOMETHANE PROCESS

(71) Applicant: Honeywell International Inc., Charlotte, NC (US)

(72) Inventors: Daniel C. Merkel, Orchard Park, NY (US); Haluk Kopkalli, Staten Island, NY (US); Haiyou Wang, Amherst, NY (US); Terris Yang, East Amherst, NY (US)

(21) Appl. No.: 18/221,968

(22) Filed: Jul. 14, 2023

Related U.S. Application Data
(60) Provisional application No. 63/390,045, filed on Jul. 18, 2022.

(51) Int. Cl. C07C 17/20; C07C 17/38; C07C 17/389; C07C 17/383

(52) U.S. Cl. CPC C07C 17/206; C07C 17/38; C07C 17/389; C07C 17/383

(57) ABSTRACT

The present disclosure provides a process for producing trifluoroiodomethane (CF₃I), with a low concentration of methyl propane. Specifically, the present disclosure provides a process for producing trifluoroiodomethane (CF₃I) with an amount of methyl propane of 100 ppm or less.

### CROSS-REFERENCE TO RELATED APPLICATION

This application claims priority to U.S. Provisional Application No. 63/390,045, filed Jul. 18, 2022, the disclosure of which is herein incorporated by reference in its entirety.

### FIELD

The present disclosure relates to processes for producing trifluoroiodomethane (CF₃I) and reducing impurities therein. Specifically, the present disclosure relates to processes to produce trifluoroiodomethane and to reduce a methyl propane impurity therein.

### BACKGROUND

Trifluoroiodomethane (CF₃I), also known as perfluoromethyliodide, trifluoromethyl iodide, or iodotrifluoromethane, is a useful compound in commercial applications, as a refrigerant or a fire suppression agent, for example. Trifluoroiodomethane is a low global warming potential molecule with almost no ozone depletion potential. Trifluoroiodomethane can replace more environmentally damaging materials.

Methods of preparing trifluoroiodomethane are known. For example, U.S. Pat. No. 7,196,236 (Mukhopadhyay et al.) discloses a catalytic process for producing trifluoroiodomethane using reactants comprising a source of iodine, at least a stoichiometric amount of oxygen, and a reactant CF₃R, where R is selected from the group consisting of —COOH, —COX, —CHO, —COOR₂, AND —SO₂X, where R₂ is alkyl group and X is a chlorine, bromine, or iodine. Hydrogen iodide, which may be produced by the reaction, can be oxidized by the at least a stoichiometric amount of oxygen, producing water and iodine for economic recycling.

In another example, U.S. Pat. No. 7,132,578 (Mukhopadhyay et al.) also discloses a catalytic, one-step process for producing trifluoroiodomethane from trifluoroacetyl chloride. However, the source of iodine is iodine fluoride (IF). In contrast to hydrogen iodide, iodine fluoride is relatively unstable, decomposing above 0° C. to I₂ and IF₅. Iodine fluoride may also not be available in commercially useful quantities.

In another example, U.S. Pat. No. 10,752,565 (Nair et al.) a gas-phase process for producing trifluoroiodomethane is disclosed. The process comprises providing a reactant stream comprising hydrogen iodide and trifluoroacetyl halide selected from the group consisting of trifluoroacetyl chloride, trifluoroacetyl fluoride, trifluoroacetyl bromide, and combinations thereof, and reacting the reactant stream in the presence of a catalyst at a temperature from about 200° C. to about 600° C. to produce a product stream comprising the trifluoroiodomethane.

There is a need to develop a more efficient process that may be scaled to produce commercial quantities of trifluoroiodomethane from relatively inexpensive raw materials with reduced impurities. For example, methyl propane (aka isobutane) may be present even in purified CF₃I product, and due to its flammable nature, methods for reducing its level in CF₃I product are needed.

### SUMMARY

The present disclosure provides an integrated process for producing trifluoroiodomethane (CF₃I) with a low concentration of impurities.

According to one embodiment, the present disclosure provides a process for producing trifluoroiodomethane (CF₃I), the process including: (a) providing a first reactant stream comprising hydrogen iodide (HI); (b) reacting the first reactant stream with a second reactant stream comprising trifluoroacetyl chloride (TFAC) to produce an intermediate product stream comprising trifluoroacetyl iodide (TFAI); and (c) reacting the intermediate product stream to produce a final product stream comprising trifluoroiodomethane (CF₃I).

### BRIEF DESCRIPTION OF THE DRAWINGS

FIG. 1 shows an example embodiment of a process for making CF₃I from HI and TFAC.

FIG. 2 shows an alternate embodiment of methyl propane removal from CF₃I (“post-removal”).

FIG. 3 shows an embodiment of methyl propane removal from TFAI using a solvent.

### DETAILED DESCRIPTION

The present disclosure provides a process for producing trifluoroiodomethane (CF₃I) with a low concentration of methyl propane (2-methyl propane) according to the overall reaction scheme below:

H₂+I₂→2HI Eq. 1:

TFAC+HI→TFAI+HCl Eq. 2:

TFAI→CF₃I+CO Eq. 3:

The formation of methyl propane (2-methyl propane) may have an effect on the overall yield of the reaction as well as causing difficulties in purification and is therefore undesirable. Methyl propane may form an azeotrope-like composition or a tangent pinch as disclosed in published papers by Guo, et al. and Maalem, et al. which are herein incorporated by reference. In practical terms, this behavior between methyl propane and trifluoroiodomethane (CF₃I), makes it difficult to remove methyl propane without a substantial loss in desired product yield.

An “azeotrope-like” composition is a composition of two or more components which behaves substantially as an azeotrope composition. Thus, for the purposes of this disclosure, an azeotrope-like composition is a combination of two or more different components which, when in liquid form under given pressure, will boil at a substantially constant temperature, and which will provide a vapor composition substantially identical to the liquid composition undergoing boiling.

A “tangent pinch” may be defined as a liquid mixture of two or more substances in which the relative volatility of the components is so close as to make distillation impractical. A tangent pinch or a near-azeotrope occurs when the relative volatility between the components to be separated is below 1.05 (Perry's Chemical Engineers' Handbook, 8th ed).

As used herein, the term “consisting essentially of”, with respect to the components of an azeotrope or azeotrope-like composition or mixture, means the composition contains the indicated components in an azeotrope or azeotrope-like ratio, and may contain additional components provided that the additional components do not form new azeotrope or azeotrope-like systems. For example, azeotrope mixtures consisting essentially of two compounds are those that form binary azeotropes, which optionally may include one or more additional components, provided that the additional components do not render the mixture non-azeotropic and do not form an azeotrope with either or both of the compounds (e.g., do not form a ternary or higher azeotrope).

Without wishing to be bound by theory, it is thought that methyl propane forms from iodomethane and iodopropane during the second step (Eq. 2) of the reaction. Iodomethane and iodopropane may be present as impurities in the hydrogen iodide (HI) produced in the first reaction step (Eq. 1). It is theorized that the iodomethane and iodopropane are produced from residual hydrocarbons in the incoming iodine. The hydrocarbons are theorized to be artifacts of the iodine recovery process from natural sources.

The present disclosure provides a process for producing trifluoroiodomethane (CF₃I) with a low concentration of methyl propane by reacting a first reactant stream comprising hydrogen iodide (HI) with a second reactant stream comprising trifluoroacetyl chloride (TFAC) to produce an intermediate product stream comprising trifluoroacetyl iodide (TFAI). The intermediate product stream may be purified to remove methyl propane, and then reacted to produce a final product stream comprising trifluoroiodomethane (CF₃I). Methods to remove methyl propane from trifluoroacetyl iodide (TFAI) include, but are not limited to distillation, solvent extraction, extractive distillation; adsorption via molecular sieves, carbon, carbon molecular sieves, zeolites, etc. and combinations of these methods.

In addition to purifying the trifluoroacetyl iodide (TFAI), the present disclosure provides a method to reduce methyl propane formation by removing both iodomethane and iodopropane impurities from the HI feed via distillation.

Using the methods of the present disclosure to remove methyl propone and its precursors from the feed streams provides the final product, trifluoroiodomethane (CF₃I), with a low concentration of methyl propane.

Finally, the present disclosure also provides methods to remove methyl propane from trifluoroiodomethane (CF₃I) including but not limited to distillation, solvent extraction, extractive distillation; adsorption via molecular sieves, carbon, carbon molecular sieves, zeolites, etc. and combinations of these methods.

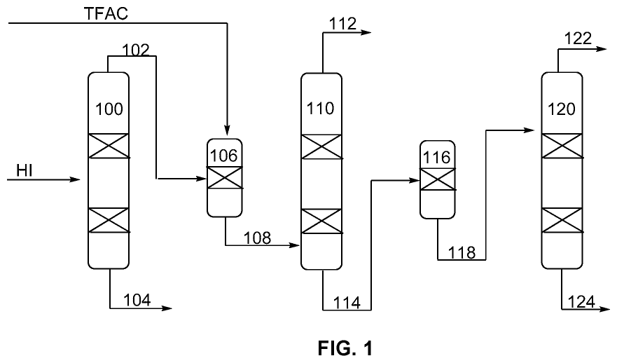

As shown in FIG. 1, a stream of HI is passed to a first distillation column 100 to provide a first bottoms product 104 comprising iodomethane and iodopropane, and a first overhead product 102 comprising purified HI. The purified HI may be passed to a first reactor 106 and reacted with a stream of TFAC in the presence of a catalyst to provide an intermediate product stream 108 comprising crude TFAI. The crude TFAI 108 may be passed to a second distillation column 110 to provide a second overhead product 112 comprising methyl propane and a second bottoms product 114 comprising purified TFAI. Purified TFAI 114 may then be passed to a second reactor 116 to provide a product stream 118 comprising crude CF₃I. Crude CF₃I 118 may then be passed to a third distillation column 120 to provide a third overhead product 122 comprising purified CF₃I and a third bottoms product 124 comprising methyl propane.

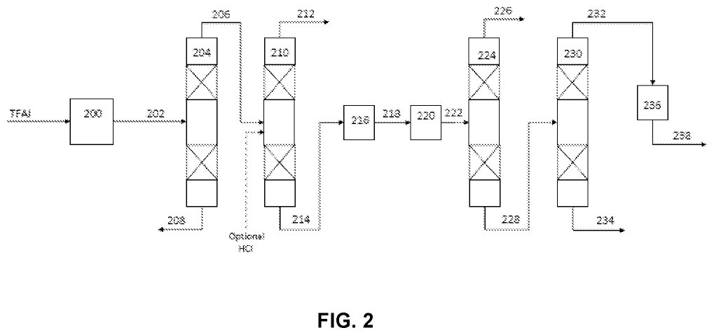

FIG. 2 shows an alternate embodiment of methyl propane removal from CF₃I (“post-removal”). In this embodiment, a stream comprising TFAI is fed to a reactor 200 to provide a product stream 202 comprising crude CF₃I, methyl propane and unreacted TFAI. The crude CF₃I 202 may be passed to a first distillation column 204 to provide a first overhead product 206 comprising CF₃I and methyl propane and a first bottoms product 208 comprising unreacted TFAI for subsequent treatment and recycle to reactor 200. Overhead product 206 of first distillation column 204 may be passed to a second distillation column 210 to provide a second overhead product comprising CO and a bottoms product 214 comprising CF₃I and methyl propane. An optional stream of HCl gas may be fed to second distillation column 210 to facilitate economical removal of CO in overhead product 212. Bottoms product 214 may be passed through a scrubber 216 to provide an acid-free and acid halide-free stream 218 which is passed through a water removal system 220 to yield a dry and acid free stream 222 comprising CF₃I and methyl propane. The water removal system may comprise concentrated sulfuric acid or solid desiccants which are well known in the art. Stream 222 comprising CF₃I and methyl propane may be passed into a third distillation column 224 to remove an overhead stream 226 comprising light impurities and a bottoms stream 228 comprising CF₃I and methyl propane which is fed to a fourth distillation column 230. The overhead product 232 from distillation column 230 comprising CF₃I and methyl propane may be fed to an adsorbent bed 236 to selectively adsorb methyl propane yielding a purified CF₃I stream 238 which comprises a lower concentration of methyl propane. The bottoms product 234 from the fourth distillation column comprises heavies (higher boiling compounds) which are treated further.

Further referring to FIG. 2, in yet other embodiments, the unit shown as 236 may be replaced with one or more of the following: distillation, solvent extraction, or extractive distillation.

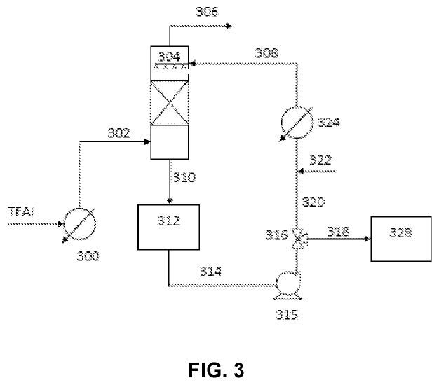

FIG. 3 shows an embodiment of methyl propane removal from TFAI using a solvent. In this embodiment, a stream comprising TFAI is fed to a vaporizer 300 to provide a vapor stream 302 which is fed to the bottom of a packed or trayed column 304. A stream of liquid solvent 308 such as n-octane, chlorobenzene or, chloroform is fed to the top of column 304. A vapor stream 306 comprising TFAI with reduced concentration of methyl propane is recovered for further processing. A stream 310 comprising the solvent and methyl propane is recovered in vessel 312. A stream 314 richer in methyl propane is optionally recirculated via pump 315 until it is saturated or near saturation. An optional purge stream rich in methyl propane may be removed via stream 318 either continuously or intermittently by opening/closing valve 316. Recirculated stream 320 may be combined with fresh solvent 322 and optionally heated via heater 324. The solvent stream 318 which is richer in methyl propane may optionally be treated in unit operation 330 to regenerate the solvent for re-use, for example, by distillation.

1. Formation of Hydrogen Iodide (HI)

As disclosed herein, in a first reaction step for the integrated process to produce CF₃I, hydrogen (H₂) may be reacted with iodine (I₂) to form hydrogen iodide (HI). The HI may be anhydrous hydrogen iodide, which is produced from a reactant stream comprising hydrogen and iodine. The reactant stream may consist essentially of hydrogen and iodine. The reactant stream may consist of hydrogen and iodine.

The production of anhydrous HI (Eq. 1) is described in greater detail below. Alternatively, HI may be produced by other means or purchased for use in the process of the invention. HI may be further purified before being fed to the integrated process to manufacture trifluoroiodomethane (CF₃I) from trifluoroacetyl chloride (TFAC) and HI.

The process includes providing a vapor-phase reactant stream comprising hydrogen and iodine and reacting the reactant stream in the presence of a catalyst to produce a product stream comprising hydrogen iodide. The catalyst includes at least one selected from the group of nickel, nickel iodide (NiI₂), cobalt, cobalt iodide (CoI₂), iron, iron iodide (FeI₂ or FeI₃), nickel oxide, cobalt oxide, and iron oxide. The catalyst may be supported on a support.

The process includes the steps of reacting hydrogen and iodine in the vapor phase in the presence of a catalyst to produce a product stream comprising HI, unreacted iodine and unreacted hydrogen, removing at least some of the unreacted iodine from the product stream by cooling the product stream to form solid iodine, producing liquid iodine from the solid iodine, and recycling the liquified iodine to the reacting step. The solid iodine forms in a first iodine removal vessel or a second iodine removal vessel. The liquid iodine is produced from the solid iodine by heating the first iodine removal vessel to liquefy the solid iodine when cooling the product stream through the second iodine removal vessel or heating the second iodine removal vessel to liquefy the solid iodine when cooling the product stream through the first iodine removal vessel. Unreacted hydrogen is recycled to the reacting step. The catalyst includes at least one selected from the group of nickel, nickel iodide (NiI₂), cobalt, cobalt iodide (CoI₂), iron, iron iodide (FeI₂ or FeI₃), nickel oxide, cobalt oxide, and iron oxide. The catalyst may be supported on a support.

The catalyst may be supplied in a passivated form and may then be activated. Additionally, the catalyst may be converted from one species to another over the course of the reaction. For example, metallic nickel on a support may be converted in situ into nickel iodide (NiI₂). Metallic nickel supported on inert materials may be commercially available at various loadings of the nickel metal. When supplied, the nickel supported on inert material is in a passivated form and may need to be activated in hydrogen gas to expose the metallic nickel phase, before iodine vapors are supplied to convert the metallic nickel phase into NiI₂. Alternatively, catalysts that may be prepared in situ like NiI₂, or may be supplied in a ready-made form, by preparing the catalyst through impregnating, pore filling, precipitation, and/or adsorption onto the support.

The catalyst may be deliquescent and when exposed to ambient conditions it may absorb moisture and dissolve in its own water of hydration. Therefore, whether the catalyst is prepared in situ or externally, it may be desired to use the catalyst in anhydrous conditions, as exposure of the catalyst to moisture may result in the formation of a hydrated complex. The formation of hydrated complexes may be associated with significant agglomeration and loss of catalytic activity. The deactivated catalyst may be regenerated by drying in hot inert gas, followed by successive repeated cycles of reduction in hydrogen gas or other suitable reducing agents and oxidation in oxygen gas or other oxidizing agents.

The catalyst may also be regenerated after a period of use. The regenerated catalyst may have reduced catalyst particle size and may have increased catalytic activity compared to spent and/or agglomerated catalyst. For example, a fresh catalyst may have a first particle size, and a spent catalyst may have a second particle size larger than the first particle size. The spent catalyst may also agglomerate when exposed to ambient conditions forming a third particle size larger than the second particle size. The agglomerated catalyst may be dried and chemically reduced to reduce the particle size and increase catalytic activity. The catalyst may undergo multiple rounds of reduction, oxidation, and drying to further reduce particle size and/or increase catalytic activity, thereby making a regenerated catalyst with a fourth particle size. The reduction may be carried out with hydrogen gas, and the oxidation may be carried out with oxygen gas. Other reducing and oxidizing agents may also be used. Suitable non-limiting examples of reducing agents include hydrogen, carbon monoxide (CO), ammonia (NH₃), alkanes such as methane (CH₄). Suitable non-limiting examples of oxidizing agents include oxygen (O₂), ozone (O₃), nitrogen oxides (NO₂, N₂O).

The hydrogen and iodine are anhydrous. It is preferred that there be as little water in the reactant stream as possible because the presence of moisture results in the formation of hydroiodic acid, which is corrosive and can be detrimental to equipment and process lines. In addition, recovery of the HI from the hydroiodic acid adds to the manufacturing costs.

The hydrogen is substantially free of water, including any water by weight in an amount less than about 500 ppm, about 300 ppm, about 200 ppm, about 100 ppm, about 50 ppm, about 30 ppm, about 20 ppm, about 10 ppm, about 5 ppm, about 2 ppm, or about 1 ppm, or less than any value defined between any two of the foregoing values. Preferably, the hydrogen comprises any water by weight in an amount less than about 50 ppm. For example, the hydrogen may comprise water in an amount of 40 ppm or less, 30 ppm or less, 20 ppm or less, 10 ppm or less, or within any range defined between any of the two foregoing values. More preferably, the hydrogen comprises any water by weight in an amount less than about 10 ppm. For example, the hydrogen may comprise water in an amount of 9 ppm or less, 8 ppm or less, 7 ppm or less, 6 ppm or less, 5 ppm or less, 4 ppm or less, 3 ppm or less, 2 ppm or less, 1 ppm or less, or within any range defined between any of the two foregoing values. Most preferably, the hydrogen comprises any water by weight in an amount less than about 5 ppm. For example, the hydrogen may comprise water in an amount of 4 ppm or less, 3 ppm or less, 2 ppm or less, 1 ppm or less, or within any range defined between any of the two foregoing values.

The iodine is also substantially free of water, and water, if present, is present in an amount of at least 1 ppm by weight. If water is present, it may be in an amount of 1 ppm or greater, 10 ppm or greater, 20 ppm or greater, 50 ppm or greater, 100 ppm or greater, 200 ppm or greater 500 ppm or greater, 1000 ppm or less, 1500 ppm or less, 2000 ppm or less, 2500 ppm or less, 3000 ppm or less or within any range defined between any of the two foregoing values. Preferably, the iodine comprises any water by weight in an amount less than about 100 ppm. For example, the iodine may comprise water in an amount of 90 ppm or less, 80 ppm or less, 70 ppm or less, 60 ppm or less, 50 ppm or less, 40 ppm or less, 30 ppm or less, 20 ppm or less, 10 ppm or less, or within any range defined between any of the two foregoing values. More preferably, the iodine comprises any water by weight in an amount less than about 30 ppm. For example, the iodine may comprise water in an amount of 25 ppm or less, 20 ppm or less, 15 ppm or less, 10 ppm or less, 5 ppm or less 1 ppm or less, or within any range defined between any of the two foregoing values. Most preferably, the iodine comprises any water by weight in an amount less than about 10 ppm. For example, the iodine may comprise water in an amount of 9 ppm or less, 8 ppm or less, 7 ppm or less, 6 ppm or less, 5 ppm or less, 4 ppm or less, 3 ppm or less, 2 ppm or less, 1 ppm or less, or within any range defined between any of the two foregoing values.

Elemental iodine in solid form is commercially available from, for example, SQM, Santiago, Chile, or Kanto Natural Gas Development Co., Ltd, Chiba, Japan. Hydrogen in compressed gas form is commercially available from, for example, Airgas, Radnor, PA.

The reactant stream and the catalyst may be pre-heated to a reaction temperature. The reaction temperature may be as low as about 150° C., about 200° C., about 250° C., about 280° C., about 290° C., about 300° C., about 310° C., or about 320° C., or to a reaction temperature as high as about 330° C., about 340° C., about 350° C., about 360° C., about 380° C., about 400° C., about 450° C., about 500° C., about 550° C., or about 600° C., or within any range defined between any two of the foregoing values, such as about 150° C. to about 600° C., about 200° C. to about 550° C., about 250° C. to about 500° C., about 280° C. to about 450° C., about 290° C. to about 400° C., about 300° C. to about 380° C., about 310° C. to about 360° C., about 320° C. to about 350° C., or about 320° C. to about 340° C., for example. Preferably, the reaction temperature is from about 200° C. to about 500° C. More preferably, the reaction temperature is from about 300° C. to about 400° C. Most preferably, the reaction temperature is from about 300° C. to about 350° C.

An operating pressure of the reactor may be as low as about 0 psig, about 10 psig, about 20 psig, about 40 psig, about 100 psig, about 125 psig, about 150 psig, about 175 psig, or about as high as 200 psig, about 250 psig, about 300 psig, about 400 psig, about 500 psig, about 600 psig, or any range defined between any two of the foregoing values, such as about 0 psig to 600 psig, about 10 psig to about 500 psig, about 20 psig to about 400 psig, about 40 psig to about 300 psig, about 100 psig to about 250 psig, about 150 psig to about 175 psig, or about 0 psig to about 175 psig, for example. Preferably, the operating pressure of the reactor is from about 5 psig to about 300 psig. More preferably, the operating pressure of the reactor is from about 5 psig to about 150 psig. Most preferably, the operating pressure of the reactor is from about 5 psig to about 120 psig.

In the reactant stream, a mole ratio of hydrogen to iodine may be as low as about 1:1, about 1.5:1, about 2:1, about 2.5:1, about 2.7:1, or about 3:1, or as high as about 4:1, about 5:1, about 6:1, about 7:1, about 8:1, about 9:1, or about 10:1, or within any range defined between any two of the foregoing values, such as about 1:1 to about 10:1, about 2:1 to about 8:1, about 3:1 to about 6:1, about 2:1 to about 5:1, about 2:1 to about 3:1, about 2.5:1 to about 3:1, or about 2.7:1 to about 3.0:1, for example. Preferably, the mole ratio of hydrogen to iodine is from about 2:1 to about 9:1. More preferably, the mole ratio of hydrogen to iodine is from about 2.5:1 to about 8:1. Most preferably, the mole ratio of hydrogen to iodine is from about 2.5:1 to 6:1.

The reactant stream may be in contact with the catalyst for a contact time as short as about 0.1 second, about 2 seconds, about 4 seconds, about 6 seconds, about 8 seconds, about 10 seconds, about 15 seconds, about 20 seconds, about 25 seconds, or about 30 seconds, or as long as about 40 seconds, about 50 seconds, about 60 seconds, about 70 seconds, about 80 seconds, about 100 seconds, about 120 seconds, about 200 seconds, or about 1,800 seconds or within any range defined between any two of the foregoing values, such as about 0.1 seconds to about 1,800 seconds, about 2 seconds to about 120 seconds, about 4 second to about 100 seconds, about 6 seconds to about 80 seconds, about 8 seconds to about 70 seconds, about 10 seconds to about 60 seconds, about 15 seconds to about 50 seconds, about 20 seconds to about 40 seconds, about 20 seconds to about 30 seconds, about 10 seconds to about 20 seconds, or about 100 seconds to about 120 seconds, for example. Preferably, the reactant stream is in contact with the catalyst for a contact time from about 2 seconds to about 200 seconds.

The hydrogen iodide (HI) formed in the reaction may be purified by distillation to remove iodomethane and iodopropane.

The purified hydrogen iodide (HI) may comprise iodomethane in an amount of 500 ppm or less, 450 ppm or less, 400 ppm or less, 350 pm or less, 300 ppm or less, 250 ppm or less, 200 ppm or less, 150 ppm or less, 100 ppm or less, 50 ppm or less, or within any range defined between any of the two foregoing values. Preferably, the purified hydrogen iodide (HI) may comprise iodomethane in an amount of 400 ppm or less. For example, the purified hydrogen iodide (HI) may comprise iodomethane in an amount of 300 ppm or less, 200 ppm or less, 100 ppm or less, 50 ppm or less, or within any range defined between any two of the foregoing values. More preferably, the purified hydrogen iodide (HI) may comprise iodomethane in an amount of 300 ppm or less. For example, the purified hydrogen iodide (HI) may comprise iodomethane in an amount of 200 ppm or less, 150 ppm or less, 100 ppm or less 50 ppm or less, 10 ppm or less, or within any range defined between any two of the foregoing values. Most preferably, the purified hydrogen iodide (HI) may comprise iodomethane in an amount of 200 ppm or less. For example, the purified hydrogen iodide (HI) may comprise iodomethane in an amount of 100 pm or less, 90 ppm or less, 80 ppm or less, 70 ppm or less, 60 ppm or less, 50 ppm or less, 40 ppm or less, 30 ppm or less, 20 ppm or less, 10 ppm or less, 5 ppm or less 1 ppm or less, or within any range defined between any of the two foregoing values.

The purified hydrogen iodide (HI) may comprise iodopropane in an amount of 500 ppm or less, 450 ppm or less, 400 ppm or less, 350 pm or less, 300 ppm or less, 250 ppm or less, 200 ppm or less, 150 ppm or less, 100 ppm or less, 50 ppm or less, or within any range defined between any of the two foregoing values. Preferably, the purified hydrogen iodide (HI) may comprise iodopropane in an amount of 400 ppm or less. For example, the purified hydrogen iodide (HI) may comprise iodopropane in an amount of 300 ppm or less, 200 ppm or less, 100 ppm or less, 50 ppm or less, or within any range defined between any two of the foregoing values. More preferably, the purified hydrogen iodide (HI) may comprise iodopropane in an amount of 300 ppm or less. For example, the purified hydrogen iodide (HI) may comprise iodopropane in an amount of 200 ppm or less, 150 ppm or less, 100 ppm or less 50 ppm or less, 10 ppm or less, or within any range defined between any two of the foregoing values. Most preferably, the purified hydrogen iodide (HI) may comprise iodopropane in an amount of 200 ppm or less. For example, the purified hydrogen iodide (HI) may comprise iodopropane in an amount of 100 pm or less, 90 ppm or less, 80 ppm or less, 70 ppm or less, 60 ppm or less, 50 ppm or less, 40 ppm or less, 30 ppm or less, 20 ppm or less, 10 ppm or less, 5 ppm or less 1 ppm or less, or within any range defined between any of the two foregoing values.

2. Formation of Trifluoroacetyl Iodide (TFAI)

As disclosed herein, in a second reaction step, TFAC (trifluoroacetyl chloride) is reacted with HI (hydrogen iodide) to form an intermediate product stream comprising TFAI (trifluoroacetyl iodide) and HCl (hydrogen chloride) according to Eq. 2:

TFAC+HI→TFAI+HCl

The process may be conducted in gas phase, liquid phase, or gas/liquid phase. The process comprises providing a reactant stream comprising hydrogen iodide and at least one trifluoroacetyl halide selected from trifluoroacetyl chloride (TFAC), trifluoroacetyl fluoride (TFAF), trifluoroacetyl bromide (TFAB), and combinations thereof, to produce an intermediate product stream comprising the trifluoroacetyl iodide (TFAI).

The process may be conducted in a reactor, such as a heated tube reactor comprising a tube made of a metal such as carbon steel, stainless steel, nickel, and/or a nickel alloy, such as a nickel-chromium alloy, a nickel-molybdenum alloy, a nickel-chromium-molybdenum alloy, or a nickel-copper alloy. Alternatively, the reactor may be constructed of a metal lined with glass or polymers such as polytetrafluoroethylene (PTFE), perfluoroalkoxy alkanes (PFA), fluorinated ethylene propylene (FEP) and other fluoropolymers. The reactor may be heated, or the feed materials may be preheated before entering the reactor. The reactor may be any type of packed bed reactor including multiple reactors in series or in parallel and including any heat exchangers in between reactors to manage heat effects.

The hydrogen iodide and the trifluoroacetyl chloride in the reactant stream may optionally react in the presence of a catalyst contained within the reactor. When the catalyst is used, it may be selected from the group comprising activated carbon, meso carbon, stainless steel, nickel, nickel-chromium alloy, nickel-chromium-molybdenum alloy, nickel-copper alloy, copper, alumina, platinum, palladium, or carbides, such as metal carbides, such as iron carbide, molybdenum carbide and nickel carbide, and non-metal carbides, such as silicon carbide, or combinations thereof. The catalyst may be in the form of a mesh, pellet, or sphere, contained within the reactor.

The reaction temperatures may be as low as about 0° C. or higher, about 25° C. or higher, about 35° C. or higher, about 40° C. or higher, about 50° C. or higher, or about 60° C. or lower, about 90° C. or lower, about 120° C. or lower, about 150° C. or lower, or about 200° C. or lower, or about 250° C. or lower, or any value encompassed by these endpoints.

The reaction pressure may be about 0 psig or higher, about 25 psig or higher, about 50 psig or higher, about 50 psig or higher, about 100 psig or higher, about 150 psig or higher, about 200 psig or higher, about 250 psig or higher, about 300 psig or lower, about 350 psig or lower, about 400 psig or lower, about 450 psig or lower, about 500 psig or lower, or any value encompassed by these endpoints.

The reactant stream may be in contact with the catalyst for a contact time of about 0.1 seconds or longer, about 0.5 seconds or longer, about 1 second or longer, about 2 seconds or longer, about 3 seconds or longer, about 5 seconds or longer, about 8 seconds or longer, about 10 seconds or longer, about 12 seconds or longer, or about 15 or longer, about 18 seconds or longer, about 20 seconds or shorter, about 25 seconds or shorter, about 30 seconds or shorter, about 35 seconds or shorter, about 40 seconds or shorter, about 50 seconds or shorter, about 60 seconds or shorter, about 80 seconds or shorter, or about 300 seconds shorter, or about 1800 seconds shorter, or any value encompassed by these endpoints.

In one embodiment, the TFAC/HI mole ratio may be as low as about 1:10, about 1:5, about 1:2, about 1:1, about 2:1, about 3:1, about 4:1, or as high as about 5:1, about 6:1, about 7:1, about 8:1, about 9:1, or about 10:1, or within any range defined between any two of the foregoing values. Preferably, the TFAC/HI ratio is from 1:2 to 2:1. More preferably, the TFAC/HI ratio is from 1:1 to 2:1.

The mixture may be vaporized and superheated to perform the reaction.

Trifluoroacetyl iodide compositions useful as a feed stock for producing trifluoroiodomethane according to the process of Equation 3 above include trifluoroacetyl iodide, at least one organic impurity comprising at least one of: difluoroiodomethane, pentafluoroiodoethane, iodomethane, iodopropane, dichlorotetrafluoroethane, dichlorotrifluoroethane, trichlorotrifluoroethane, methyltrifluoroacetate, trifluoroacetic anhydride, difluorobutane and methyl propane, and at least one inorganic impurity comprising at least one of: hydrogen iodide, hydrogen chloride, iodine and hydrogen triiodide.

The composition of the organic compounds in the intermediate product stream may be measured by gas chromatography (GC) and gas chromatography-mass spectroscopy (GC-MS) analyses. Peak areas provided by the GC analysis for each of the organic compounds can be combined to provide a GC area percentage (GC area %) of the total organic compounds for each of the organic compounds as a measurement of the relative concentrations of the organic compounds in the intermediate product stream. The GC area % may be interpreted as equivalent to a weight %.

The concentration of trifluoroacetyl iodide in the intermediate product stream, in GC area % of total organic compounds, where the reaction stream has a reaction temperature at or below about 120° C., may be as low as about 10%, about 20%, about 30%, about 35%, about 40%, about 45%, about 50%, about 55%, about 60%, about 65% or about 70%, or may be as high as about 75%, about 80%, about 85%, about 90%, about 95%, about 97%, about 98%, or about 99% or within any range defined between any two of the foregoing values, such as about 10% to about 99%, about 10% to about 99%, about 30% to about 99%, about 35% to about 98%, about 40% to about 97%, about 45% to about 95%, about 50% to about 90%, about 55% to about 85%, about 60% to about 80%, about 65% to about 75%, about 50% to about 60%, about 90% to about 99% or about 95% to about 99%, for example. Preferably, the concentration of trifluoroacetyl iodide in the intermediate product stream may be from about 50% to about 99%. More preferably, the concentration of trifluoroacetyl iodide in the intermediate product stream may be from about 60% to about 99%. Most preferably, the concentration of trifluoroacetyl iodide in the intermediate product stream may be from about 70% to about 99%.

The concentration of trifluoroiodomethane in the intermediate product stream, in GC area % of total organic compounds, where the reaction stream has a reaction temperature at or below about 120° C., may be less than about 0.010%, less than about 0.005%, less than about 0.002%, less than about 0.001%, less than about 0.0005%, less than about 0.0002%, or less than about 0.0001%, or less than any value defined between any two of the foregoing values. Preferably, the concentration of trifluoroiodomethane in the intermediate product stream may be less than about 0.002%. More preferably, the concentration of trifluoroiodomethane in the intermediate product stream may be less than about 0.001%. Most preferably, the concentration of trifluoroiodomethane in the intermediate product stream may be less than about 0.0005%.

Alternatively stated, organic compounds in the intermediate product stream, where the reaction stream has a first reaction temperature at or below about 120° C., may comprise, in GC area % of total organic compounds, from about 10% to about 99% trifluoroacetyl iodide, from about 1% to about 90% unreacted trifluoroacetyl halide, less than about 0.010% trifluoroiodomethane, and less than about 15% organic compounds other than trifluoroacetyl iodide, trifluoroacetyl halide, and trifluoroiodomethane. It is also provided that organic compounds in the intermediate product stream may comprise from about 50% to about 99% trifluoroacetyl iodide, from about 1% to about 50% unreacted trifluoroacetyl halide, less than about 0.002% trifluoroiodomethane, and less than about 8% organic compounds other than trifluoroacetyl iodide, trifluoroacetyl halide, and trifluoroiodomethane. It is also provided that organic compounds in the intermediate product stream may comprise from about 60% to about 99% trifluoroacetyl iodide, from about 1% to about 40% unreacted trifluoroacetyl halide, less than about 0.001% trifluoroiodomethane, and less than about 4% organic compounds other than trifluoroacetyl iodide, trifluoroacetyl halide, and trifluoroiodomethane. It is also provided that organic compounds in the intermediate product stream may comprise from about 70% to about 99% trifluoroacetyl iodide, from about 1% to about 30% unreacted trifluoroacetyl halide, less than about 0.0005% trifluoroiodomethane, and less than about 2% organic compounds other than trifluoroacetyl iodide, trifluoroacetyl halide, and trifluoroiodomethane.

Alternatively stated, organic compounds in the intermediate product stream, where the reaction stream has a first reaction temperature at or below about 120° C., may consist essentially of, in GC area % of total organic compounds, from about 10% to about 99% trifluoroacetyl iodide, from about 1% to about 90% unreacted trifluoroacetyl halide, less than about 0.010% trifluoroiodomethane, and less than about 15% organic compounds other than trifluoroacetyl iodide, trifluoroacetyl halide, and trifluoroiodomethane. It is also provided that organic compounds in the intermediate product stream may consist essentially of from about 50% to about 99% trifluoroacetyl iodide, from about 1% to about 50% unreacted trifluoroacetyl halide, less than about 0.002% trifluoroiodomethane, and less than about 8% organic compounds other than trifluoroacetyl iodide, trifluoroacetyl halide, and trifluoroiodomethane. It is also provided that organic compounds in the intermediate product stream may consist essentially of from about 60% to about 99% trifluoroacetyl iodide, from about 1% to about 40% unreacted trifluoroacetyl halide, less than about 0.001% trifluoroiodomethane, and less than about 4% organic compounds other than trifluoroacetyl iodide, trifluoroacetyl halide, and trifluoroiodomethane. It is also provided that organic compounds in the intermediate product stream may consist essentially of from about 70% to about 99% trifluoroacetyl iodide, from about 1% to about 30% unreacted trifluoroacetyl halide, less than about 0.0005% trifluoroiodomethane, and less than about 2% organic compounds other than trifluoroacetyl iodide, trifluoroacetyl halide, and trifluoroiodomethane.

Alternatively stated, organic compounds in the intermediate product stream, where the reaction stream has a first reaction temperature at or below about 120° C., may consist of, in GC area % of total organic compounds, from about 10% to about 99% trifluoroacetyl iodide, from about 1% to about 90% unreacted trifluoroacetyl halide, less than about 0.010% trifluoroiodomethane, and less than about 15% organic compounds other than trifluoroacetyl iodide, trifluoroacetyl halide, and trifluoroiodomethane. It is also provided that organic compounds in the intermediate product stream may consist of from about 50% to about 99% trifluoroacetyl iodide, from about 1% to about 50% unreacted trifluoroacetyl halide, less than about 0.002% trifluoroiodomethane, and less than about 8% organic compounds other than trifluoroacetyl iodide, trifluoroacetyl halide, and trifluoroiodomethane. It is also provided that organic compounds in the intermediate product stream may consist of from about 60% to about 99% trifluoroacetyl iodide, from about 1% to about 40% unreacted trifluoroacetyl halide, less than about 0.001% trifluoroiodomethane, and less than about 4% organic compounds other than trifluoroacetyl iodide, trifluoroacetyl halide, and trifluoroiodomethane. It is also provided that organic compounds in the intermediate product stream may consist of from about 70% to about 99% trifluoroacetyl iodide, from about 1% to about 30% unreacted trifluoroacetyl halide, less than about 0.0005% trifluoroiodomethane, and less than about 2% organic compounds other than trifluoroacetyl iodide, trifluoroacetyl halide, and trifluoroiodomethane.

In the manufacture of trifluoroiodomethane according to Equation 3, the trifluoroacetyl iodide is provided as a composition including organic and inorganic impurities because pure trifluoroacetyl iodide can be too costly to permit the economically efficient manufacture of trifluoroiodomethane. It has been found that some impurities can have a more detrimental impact on the overall efficiency of process than other impurities. For example, it is believed that some hydrogen-containing impurities and some halogen-containing impurities in the trifluoroacetyl iodide composition can result in increased formation of byproducts, such as methyl trifluoride (CF₃H) and iodine (I₂). The production of these byproducts comes at the expense of the production of the desired trifluoroiodomethane product. Examples of hydrogen-containing impurities include trifluoroacetic acid (TFA), hydrogen iodide (HI), hydrogen chloride (HCl) and hydrogen triiodide (HI₃). Examples of halogen-containing impurities include trifluoroacetyl fluoride (CF₃COF), trifluoroacetyl chloride (CF₃COCl), difluoroiodomethane (CF₂HI), pentafluoroiodoethane (CF₃CF₂I), iodomethane (CH₃I), iodine (I₂), iodopropane (C₃H₇I) and trifluoroiodomethane (CF₃I). While the trifluoroiodomethane is the desired product, trifluoroiodomethane in the trifluoroacetyl iodide composition feeding the reaction can react to produce undesired byproducts.

It is also believed that increased concentrations of some iodine-containing impurities, such as iodine (I₂) and hydrogen triiodide (HI₃), can cause increased corrosion of processing equipment. The increased corrosion can reduce the useful lifetime of the processing equipment.

In contrast, it has been found that some other organic impurities in the trifluoroacetyl iodide composition have relatively little effect on the efficiency of the process. Such impurities generally pass through the reactor without reacting and do not corrode the processing equipment. Examples of such organic impurities include dichlorotetrafluoroethane (C₂Cl₂F₄), dichlorotrifluoroethane (C₂HCl₂F₃), chlorotrifluoroethane (C₂H₂ClF₃), trichlorotrifluoroethane (C₂Cl₃F₃), methyltrifluoroacetate (CF₃COOCH₃), trifluoroacetic anhydride ((CF₃CO)₂O), difluorobutane (C₄H₈F₂) and methyl propane (CH₃CH(CH₃)CH₃). However, these impurities reduce the assay and therefore need to be removed.

3. Purification of TFAI

TFAI may be purified to remove the majority of all of the methyl propane by distillation, gas/liquid extraction with a solvent, extractive distillation, mole sieves, carbon, and carbon molecular sieves, and combination of these methods. Solvents used in extractions may be treated to regenerate the solvent for re-use, for example, by distillation. Generally, any of these methods may performed as a batch or continuous process.

Suitable molecular sieves may include 3 Å molecular sieves available from Acros Organics (also available from Honeywell UOP); 4 Å and XH-9 molecular sieves available from Honeywell UOP, 10 Å molecular sieves available from Grace Davison, and carbon molecular sieves, such as MSC-3K 172 carbon molecular sieves available from Osaka Gas Chemicals; activated alumina, such as SAS40 ⅛″ Alumina available from BASF; zeolite ammonium powders, such as CBV5524G CY, Zeolite 13X, and Zeolite ZSM-5 available from Zeolyst International; and activated charcoal, such as NORIT ROX 0.8 Activated Carbon and BAX 1500 activated carbon available from Cabot.

Suitable gas liquid extraction solvents include, for example, hydrocarbons, especially n-octane, chlorinated compounds, especially chlorobenzene or chloroform.

TFAI may be purified by distillation to remove methyl propane, along with other impurities. A batch distillation column comprising a reboiler, shell and tube condenser, and packed column may be used. A lights cut may be removed, comprising CF₃I, TFAI, and methyl propane. A continuous distillation scheme involving two distillation columns may also be used. One column will be used to remove impurities that are lower boiling than TFAI such as CF₃I and methyl propane and some amount of TFAI. A second column will be used to separate higher boiling impurities from TFAI.

During the lights cut of a typical batch distillation, the column overhead temperature may be about 0° C. or greater, about 5° C. or greater, about 10° C. or greater, about 15° C. or less, about 20° C. or less, about 25° C. or less, about 30° C. or less, or any value or range encompassed by these endpoints, independent of pressure and composition.

Following the removal of lights, the main distillate cut may begin. The distillation is considered complete when the amount of undesired high boiling impurities in the distillate is greater than desired.

During the main cut of a typical batch distillation, the column overhead temperature may be about 10° C. or greater, about 15° C. or greater, about 20° C. or greater, about 25° C. or less, about 30° C. or less, about 35° C. or less, about 40° C. or less, or any value or range encompassed by these endpoints, independent of pressure and composition.

Following purification, the concentration of TFAI in the TFAI stream may be about 90% or greater, about 91% or greater, about 92% or greater, about 93% or greater, about 94% or greater, about 95% or greater, about 96% or greater, about 97% or greater, about 98% or greater, or about 99% or greater, as measured by gas chromatography/mass spectrometry (GC/MS).

The concentration of the trifluoroacetyl iodide in the purified intermediate product stream may be greater than about 98 weight percent (wt. %). Preferably, the concentration of the trifluoroacetyl iodide in the purified intermediate product stream may be greater than about 99 wt. %. More preferably, the concentration of the trifluoroacetyl iodide in the purified intermediate product stream may be greater than about 99.5 wt. %. Most preferably, the concentration of the trifluoroacetyl iodide in the purified intermediate product stream may be greater than about 99.7 wt. %.

The concentration of organic impurities in the trifluoroacetyl iodide compositions, in GC area % of total organic compounds, may be as low as about 0.05%, about 0.1%, about 0.5%, or about 1% or may be as high as about 2%, about 3%, about 4%, or about 5%, or within any range defined between any two of the foregoing values, such as about 0.05% to about 5%, about 0.1% to about 4%, about 0.5% to about 3%, about 1% to about 2%, about 0.05% to about 2%, about 0.5% to about 2%, about 2% to about 5%, or about 0.05% to about 1%, for example. Preferably, the concentration of organic impurities in the trifluoroacetyl iodide compositions, in GC area % of total organic compounds, may be from about 0.05% to about 3%. More preferably, the concentration of organic impurities in the trifluoroacetyl iodide compositions, in GC area % of total organic compounds, may be from about 0.05% to about 2%. Most preferably, the concentration of organic impurities in the trifluoroacetyl iodide compositions, in GC area % of total organic compounds, may be from about 0.05% to about 1%.

The concentration of difluoroiodomethane, iodopropane, dichlorotetrafluoroethane, dichlorotrifluoroethane, difluorobutane and methyl propane in total in the trifluoroacetyl iodide compositions, in GC area % of total organic compounds, may be as low as about 0.0001%, about 0.001%, about 0.005%, about 0.01%, about 0.02% or about 0.03%, or may be as high as about 0.05%, about 0.1%, about 0.2%, about 0.3% or about 0.5%, or within any range defined between any two of the foregoing values, such as about 0.0001% to about 0.5%, about 0.001% to about 0.3%, about 0.005% to about 0.2%, about 0.01% to about 0.1%, about 0.001% to about 0.2%, about 0.001% to about 0.03%, about 0.05% to about 0.5%, or about 0.1% to about 0.3%, for example.

The concentration of trifluoroacetic anhydride when present in the trifluoroacetyl iodide compositions, in GC area % of total organic compounds, may be as low as about 0.001%, about 0.005%, about 0.01%, about 0.02% or about 0.03%, or may be as high as about 0.05%, about 0.1%, about 0.2%, about 0.3%, about 0.5% or about 1%, or within any range defined between any two of the foregoing values, such as about 0.001% to about 1%, about 0.005% to about 0.5%, about 0.01% to about 0.3%, about 0.02% to about 0.2%, about 0.03% to about 0.1%, about 0.01% to about 0.05%, about 0.1% to about 1%, or about 0.01% to about 0.3%, for example.

The concentration of pentafluoroiodoethane when present in the trifluoroacetyl iodide compositions, in GC area % of total organic compounds, may be as low as about 0.0001%, about 0.001%, about 0.005%, about 0.01%, about 0.02% or about 0.03%, or may be as high as about 0.05%, about 0.1%, about 0.2%, about 0.3% or about 0.5%, or within any range defined between any two of the foregoing values, such as about 0.0001% to about 0.5%, about 0.001% to about 0.3%, about 0.005% to about 0.2%, about 0.01% to about 0.1%, about 0.001% to about 0.2%, about 0.001% to about 0.03%, about 0.05% to about 0.5%, or about 0.1% to about 0.3%, for example. Preferably, the concentration of pentafluoroiodoethane in the trifluoroacetyl iodide compositions, in GC area % of total organic compounds, is from about 0.0001% to about 0.2%. More preferably, the concentration of pentafluoroiodoethane in the trifluoroacetyl iodide compositions, in GC area % of total organic compounds, is from about 0.0001% to about 0.1%. Most preferably, the concentration of pentafluoroiodoethane in the trifluoroacetyl iodide compositions, in GC area % of total organic compounds, is from about 0.0001% to about 0.05%.

The concentration of iodomethane when present in the trifluoroacetyl iodide compositions, in GC area % of total organic compounds, may be as low as about 0.0001%, about 0.001%, about 0.005%, about 0.01%, about 0.02% or about 0.03%, or may be as high as about 0.05%, about 0.1%, about 0.2%, about 0.3% or about 0.5%, or within any range defined between any two of the foregoing values, such as about 0.0001% to about 0.5%, about 0.001% to about 0.3%, about 0.005% to about 0.2%, about 0.01% to about 0.1%, about 0.001% to about 0.2%, about 0.001% to about 0.03%, about 0.05% to about 0.5%, or about 0.1% to about 0.3%, for example. Preferably, the concentration of iodomethane in the trifluoroacetyl iodide compositions, in GC area % of total organic compounds, is from about 0.0001% to about 0.2%. More preferably, the concentration of iodomethane in the trifluoroacetyl iodide compositions, in GC area % of total organic compounds, is from about 0.0001% to about 0.1%. Most preferably, the concentration of iodomethane in the trifluoroacetyl iodide compositions, in GC area % of total organic compounds, is from about 0.0001% to about 0.05%.

The concentration of methyltrifluoroacetate, when present, in the trifluoroacetyl iodide compositions, in GC area % of total organic compounds, may be as low as about 0.001%, about 0.005%, about 0.01%, about 0.02%, about 0.03% or about 0.05%, or may be as high as about 0.1%, about 0.2%, about 0.3%, about 0.5%, about 1%, or about 2%, or within any range defined between any two of the foregoing values, such as about 0.001% to about 2%, about 0.005% to about 1%, about 0.01% to about 0.5%, about 0.02% to about 0.3%, about 0.03% to about 0.2%, about 0.05% to about 0.1%, about 0.1% to about 1%, or about 0.02% to about 0.1%, for example.

Any of the trifluoroacetyl iodide compositions described above may further include at least one additional organic impurity comprising at least one of: trifluoroacetic acid, trifluoroacetyl fluoride, trifluoroacetyl chloride, trifluoroiodomethane and chlorotrifluoroethane.

The concentration of trifluoroacetic acid when present in the trifluoroacetyl iodide compositions, in GC area % of total organic compounds, may be as low as about 0.001%, about 0.005%, about 0.01%, about 0.02%, about 0.03% or about 0.05%, or may be as high as about 0.1%, about 0.2%, about 0.3%, about 0.5%, about 1%, or about 2%, or within any range defined between any two of the foregoing values, such as about 0.001% to about 2%, about 0.005% to about 1%, about 0.01% to about 0.5%, about 0.02% to about 0.3%, about 0.03% to about 0.2%, about 0.05% to about 0.1%, about 0.1% to about 1%, or about 0.02% to about 0.1%, for example. Preferably, the concentration of trifluoroacetic acid in the trifluoroacetyl iodide compositions, in GC area % of total organic compounds, is from about 0.001% to about 1%. More preferably, the concentration of trifluoroacetic acid in the trifluoroacetyl iodide compositions, in GC area % of total organic compounds, is from about 0.001% to about 0.5%. Most preferably, the concentration of trifluoroacetic acid in the trifluoroacetyl iodide compositions, in GC area % of total organic compounds, is from about 0.001% to about 0.2%.

The concentration of trifluoroacetyl fluoride when present in the trifluoroacetyl iodide compositions, in GC area % of total organic compounds, may be as low as about 0.0001%, about 0.0005%, about 0.001%, about 0.002% or about 0.003%, or may be as high as about 0.005%, about 0.01%, about 0.02%, about 0.03% or about 0.05%, or within any range defined between any two of the foregoing values, such as about 0.0001% to about 0.05%, about 0.0005% to about 0.03%, about 0.001% to about 0.02%, about 0.002% to about 0.01%, about 0.003% to about 0.005%, about 0.0005% to about 0.02%, about 0.005% to about 0.05%, or about 0.001% to about 0.01%, for example. Preferably, the concentration of trifluoroacetyl fluoride in the trifluoroacetyl iodide compositions, in GC area % of total organic compounds, is from about 0.0001% to about 0.02%. More preferably, the concentration of trifluoroacetyl fluoride in the trifluoroacetyl iodide compositions, in GC area % of total organic compounds, is from about 0.0001% to about 0.01%. Most preferably, the concentration of trifluoroacetyl fluoride in the trifluoroacetyl iodide compositions, in GC area % of total organic compounds, is from about 0.0001% to about 0.005%.

The concentration of trifluoroacetyl chloride when present in the trifluoroacetyl iodide compositions, in GC area % of total organic compounds, may be as low as about 0.001%, about 0.005%, about 0.01%, about 0.02%, about 0.03% or about 0.05%, or may be as high as about 0.1%, about 0.2%, about 0.3%, about 0.5%, about 1%, or about 2%, or within any range defined between any two of the foregoing values, such as about 0.001% to about 2%, about 0.005% to about 1%, about 0.01% to about 0.5%, about 0.02% to about 0.3%, about 0.03% to about 0.2%, about 0.05% to about 0.1%, about 0.1% to about 1%, or about 0.02% to about 0.1%, for example. Preferably, the concentration of trifluoroacetyl chloride in the trifluoroacetyl iodide compositions, in GC area % of total organic compounds, is from about 0.001% to about 1%. More preferably, the concentration of trifluoroacetyl chloride in the trifluoroacetyl iodide compositions, in GC area % of total organic compounds, is from about 0.001% to about 0.5%. Most preferably, the concentration of trifluoroacetyl chloride in the trifluoroacetyl iodide compositions, in GC area % of total organic compounds, is from about 0.001% to about 0.2%.

The concentration of trifluoroiodomethane when present in the trifluoroacetyl iodide compositions, in GC area % of total organic compounds, may be as low as about 0.0001%, about 0.001%, about 0.01%, about 0.02% or about 0.05%, or may be as high as about 0.1%, about 0.5%, about 1%, or about 2% or within any range defined between any two of the foregoing values, such as about 0.0001% to about 2%, about 0.001% to about 1%, about 0.01% to about 0.5%, about 0.02% to about 0.1%, about 0.001% to about 0.05%, about 0.1% to about 0.5%, about 0.05% to about 0.3%, or about 0.001% to about 0.03%, for example. Preferably, the concentration of trifluoroiodomethane in the trifluoroacetyl iodide compositions, in GC area % of total organic compounds, is from about 0.0001% to about 1%. More preferably, the concentration of trifluoroiodomethane in the trifluoroacetyl iodide compositions, in GC area % of total organic compounds, is from about 0.0001% to about 0.5%. Most preferably, the concentration of trifluoroiodomethane in the trifluoroacetyl iodide compositions, in GC area % of total organic compounds, is from about 0.0001% to about 0.1%.

The concentration of chlorotrifluoroethane when present in the trifluoroacetyl iodide compositions, in GC area % of total organic compounds, may be as low as about 0.0001%, about 0.001%, about 0.005%, about 0.01%, about 0.02% or about 0.03%, or may be as high as about 0.05%, about 0.1%, about 0.2%, about 0.3% or about 0.5%, or within any range defined between any two of the foregoing values, such as about 0.0001% to about 0.5%, about 0.001% to about 0.3%, about 0.005% to about 0.2%, about 0.01% to about 0.1%, about 0.001% to about 0.2%, about 0.001% to about 0.03%, about 0.05% to about 0.5%, or about 0.1% to about 0.3%, for example.

The concentration of iodine (I₂) in the trifluoroacetyl iodide compositions may be measured via titration, as is known in the art. The concentration of hydrogen iodide, hydrogen chloride, hydrogen triiodide and other hydrogen-containing inorganic compounds may be measured by H-NMR, as is known in the art.

The concentration of inorganic impurities in the trifluoroacetyl iodide compositions, may be as low as about 0.01 weight percent (wt. %), about 0.02 wt. %, about 0.03 wt. % or about 0.05 wt. %, or as high as about 0.1 wt. %, about 0.2 wt. %, about 0.3 wt. %, about 0.5 wt. %, about 1 wt. %, or about 1.5 wt. %, or within any range defined between any two of the foregoing values, such as about 0.01 wt. % to about 1.5 wt. %, about 0.02 wt. % to about 1 wt. %, about 0.03 wt. % to about 0.5 wt. %, about 0.05 wt. % to about 0.3 wt. %, about 0.02 wt. % to about 0.2 wt. %, about 0.01 wt. % to about 0.05 wt. %, about 0.1 wt. % to about 1 wt. %, or about 0.1 wt. % to about 0.5 wt. %, for example. Preferably, the concentration of inorganic impurities in the trifluoroacetyl iodide compositions is from about 0.01 wt. % to about 0.5 wt. %. More preferably, the concentration of inorganic impurities in the trifluoroacetyl iodide compositions is from about 0.01 wt. % to about 0.1 wt. %. Most preferably, the concentration of inorganic impurities in the trifluoroacetyl iodide compositions is from about 0.01 wt. % to about 0.05 wt. %.

The concentration of hydrogen iodide when present in the trifluoroacetyl iodide compositions, may be as low as about 0.0001 wt. %, 0.001 wt. %, about 0.005 wt. %, about 0.01 wt. %, about 0.02 wt. %, or about 0.03 wt. %, or as high as about 0.05 wt. %, about 0.1 wt. %, about 0.2 wt. %, about 0.3 wt. % or about 0.5 wt. %, or within any range defined between any two of the foregoing values, such as about 0.0001 wt. % to about 0.5 wt. %, about 0.001 wt. % to about 0.5 wt. %, about 0.005 wt. % to about 0.3 wt. %, about 0.01 wt. % to about 0.2 wt. %, about 0.02 wt. % to about 0.1 wt. %, about 0.03 wt. % to about 0.05 wt. %, about 0.01 wt. % to about 0.3 wt. %, about 0.005 wt. % to about 0.03 wt. %, about 0.05 wt. % to about 0.5 wt. %, or about 0.05 wt. % to about 0.3 wt. %, for example. Preferably, the concentration of hydrogen iodide in the trifluoroacetyl iodide compositions is from about 0.0001 wt. % to about 0.3 wt. %. More preferably, the concentration of hydrogen iodide in the trifluoroacetyl iodide compositions is from about 0.0001 wt. % to about 0.1 wt. %. Most preferably, the concentration of hydrogen iodide in the trifluoroacetyl iodide compositions is from about 0.0001 wt. % to about 0.05 wt. %.

The concentration of iodine (I₂) when present in the trifluoroacetyl iodide compositions, may be as low as about 0.001 wt. %, about 0.005 wt. %, about 0.01 wt. %, about 0.02 wt. %, or about 0.03 wt. %, or as high as about 0.05 wt. %, about 0.1 wt. %, about 0.2 wt. %, about 0.3 wt. % or about 0.5 wt. %, or within any range defined between any two of the foregoing values, such as about 0.001 wt. % to about 0.5 wt. %, about 0.005 wt. % to about 0.3 wt. %, about 0.01 wt. % to about 0.2 wt. %, about 0.02 wt. % to about 0.1 wt. %, about 0.03 wt. % to about 0.05 wt. %, about 0.01 wt. % to about 0.3 wt. %, about 0.005 wt. % to about 0.03 wt. %, about 0.05 wt. % to about 0.5 wt. %, or about 0.05 wt. % to about 0.3 wt. %, for example. Preferably, the concentration of iodine in the trifluoroacetyl iodide compositions is from about 0.001 wt. % to about 0.3 wt. %. More preferably, the concentration of iodine in the trifluoroacetyl iodide compositions is from about 0.001 wt. % to about 0.1 wt. %. Most preferably, the concentration of iodine in the trifluoroacetyl iodide compositions is from about 0.001 wt. % to about 0.05 wt. %.

The concentration of hydrogen triiodide when present in the trifluoroacetyl iodide compositions, may be as low as about 0.0001 wt. %, about 0.001 wt. %, about 0.005 wt. %, about 0.01 wt. %, about 0.02 wt. %, or about 0.03 wt. %, or as high as about 0.05 wt. %, about 0.1 wt. %, about 0.2 wt. %, about 0.3 wt. % or about 0.5 wt. %, or within any range defined between any two of the foregoing values, such as about 0.0001 wt. % to about 0.05 wt. %, about 0.001 wt. % to about 0.5 wt. %, about 0.005 wt. % to about 0.3 wt. %, about 0.01 wt. % to about 0.2 wt. %, about 0.02 wt. % to about 0.1 wt. %, about 0.03 wt. % to about 0.05 wt. %, about 0.01 wt. % to about 0.3 wt. %, about 0.005 wt. % to about 0.03 wt. %, about 0.05 wt. % to about 0.5 wt. %, or about 0.05 wt. % to about 0.3 wt. %, for example. Preferably, the concentration of hydrogen triiodide in the trifluoroacetyl iodide compositions is from about 0.0001 wt. % to about 0.3 wt. %. More preferably, the concentration of hydrogen triiodide in the trifluoroacetyl iodide compositions is from about 0.0001 wt. % to about 0.1 wt. %. Most preferably, the concentration of hydrogen triiodide in the trifluoroacetyl iodide compositions is from about 0.0001 wt. % to about 0.05 wt. %.

The concentration of methyl propane following purification when present in the trifluoroacetyl iodide compositions as measured by gas chromatography/mass spectrometry (GC/MS), may be as low as about 1 wt. % or less, about 0.5 wt. % or less, about 1000 ppm or less, about 500 ppm or less, about 100 ppm or less, about 50 ppm or less, about 10 ppm or less, or within any range defined between any of the two foregoing values.

4. Formation of Trifluoroiodomethane (CF₃I) from Trifluoroacetyl Iodide (TFAI)

As discussed above, in the third reaction step (Eq. 3), trifluoroacetyl iodide (TFAI) is reacted to form trifluoroiodomethane (CF₃I) and carbon monoxide (CO). The present disclosure provides gas-phase processes for producing trifluoroiodomethane (CF₃I).

The process comprises providing a reactant stream comprising TFAI, providing the stream to a reactor, optionally contacting the stream with a catalyst, and converting the stream in a reactor to produce a product stream comprising the CF₃I.

When a catalyst is used, the catalyst may comprise stainless steel, nickel, nickel-chromium alloy, nickel-chromium-molybdenum alloy, nickel-copper alloy, copper, alumina, silicon carbide, platinum, palladium, rhenium, activated carbon, or combinations thereof. The catalyst may comprise activated carbon.

The reaction temperature may be about 200° C. or higher, about 250° C. or higher, about 300° C. or higher, about 350° C. or higher, about 400° C. or higher, about 450° C. or lower, about 500° C. or lower, about 550° C. or lower, about 600° C. or lower, or any value encompassed by these endpoints. Preferably, the reaction may be carried out at a temperature from about 300° C. to about 500° C. More preferably, the reaction may be carried out at a temperature from about 300° C. to about 450° C.

The reaction may be carried out at a pressure of about 0 psig or greater, about 5 psig or greater, about 20 psig or greater, about 50 psig or greater, about 70 psig or greater, about 100 psig or greater, about 150 psig or lower, about 200 psig or lower, about 225 psig or lower, about 250 psig or lower, about 275 psig or lower, about 300 psig, or within any range encompassing these endpoints. However, any pressure, such as sub-atmospheric or super-atmospheric pressures may be used in the reaction.

The contact time of the reactant stream with the catalyst may be about 0.1 second or longer, about 1 second or longer, about 5 seconds or longer, about 10 seconds or longer, about 20 seconds or longer, about 30 seconds or longer, about 40 seconds or longer, about 50 seconds or less, about 60 seconds or less, about 80 seconds or less, about 100 seconds or less, about 120 seconds or less, about 180 seconds or less, or any value encompassed by these endpoints.

The process may be a continuous process. The process may further comprise the additional steps of separating unreacted TFAI from the product stream and returning the separated unreacted TFAI to the reactant stream. The process may further comprise the additional step of separating CO from the product stream. The process may further comprise the additional step of condensing and collecting CF₃I as crude product.

The concentration of CF₃I in the CF₃I crude product may be greater than 99 wt. %, such as about 99 wt. % or greater, about 99.5 wt. % or greater, or about 99.9 wt. % or greater.

The final product stream comprises a composition compromising trifluoroiodomethane and carbon monoxide by-product and unreacted trifluoroacetyl iodide, as shown in Equation 3. The final product stream composition may further include residual impurities from the purified intermediate product stream, such as trifluoroacetyl chloride (CF₃CPCl), and chlorotrifluoroethane (C₂H₂ClF₃), as well as byproducts, such as trifluoromethane (CHF₃), hexafluoroethane (C₂F₆), trifluoroacetyl fluoride (CF₃COF), hexafluoropropanone (CF₃COCF₃), trifluoroacetaldehyde (CF₃COH), trifluorochloroethane (CF₃Cl), pentafluoroiodoethane (C₂F₅I), difluoroiodomethane (CHF₂I), pentafluoropropanone (CF₃COCHF₂), trifluoroacetic acid anhydride (CF₃COOCOCF₃), heptafluoroiodopropane (C₃F₇I), iodomethane (CH₃I), methyl propane (CH₃CH(CH₃)CH₃), difluorochloroiodomethane (CClF₂I), and/or trifluoroacetic acid (CF₃COOH).

9. Purification of Trifluoroiodomethane (CF₃I)

Following reaction, the concentration of trifluoroiodomethane (CF₃I) in the crude CF₃I stream may be about 95% or greater, about 96% or greater, about 97% or greater, about 98% or greater, or about 99% or greater, as measured by gas chromatography (GC). The crude CF₃I may be subjected to batch distillation to remove some of the methyl propane and other impurities. A continuous distillation scheme involving two distillation columns may also be used. A first column may be used to remove impurities that are lower boiling than CF₃I such as carbon monoxide, trifluoromethane, chlorotrifluoromethane, trifluoroacetyl chloride, trifluoroacetyl fluoride, and hexafluoroethane along with some amount of CF₃I. A second column may be used to separate higher boiling impurities such as methyl propane and iodomethane from CF₃I.

In a first cut of a typical batch distillation, non-condensibles and lights may be removed off the top of the condenser and the purity of the stream may be monitored by GC. Once low-boiling impurities, such as trifluoromethane (CHF₃) are reduced to a concentration equal to or below the desired level in the distillate, the main cut may begin.

During the main cut of the batch distillation, the column overhead temperature may be about 10° C. or greater, about 15° C. or greater, about 16° C. or greater, about 17° C. or greater, about 18° C. or greater, about 19° C. or greater, about 20° C. or greater, about 21° C. or less, about 22° C. or less, about 23° C. or less, about 24° C. or less, about 25° C. or less, about 30° C. or less, or any value or range encompassed by these endpoints, depending upon the pressure.

The purity of the CF₃I product following purification may have a trifluoroiodomethane concentration greater than 99 wt. %. Preferably, the concentration of the trifluoroiodomethane in the purified final product composition may be greater than 99.5 wt. %. More preferably, the concentration of the trifluoroiodomethane in the purified final product composition may be greater than 99.7 wt. %. Most preferably, the concentration of trifluoroiodomethane in the purified final product composition may be greater than 99.9 wt. %. And even more preferably, the concentration of trifluoroiodomethane in the purified final product composition may be greater than 99.99 wt. %, as measured by GC FID area %.

The concentration of some impurities in the purified final product stream may detract from the performance of the trifluoroiodomethane and its intended purpose as an environmentally safe, non-toxic gas. If the trifluoroacetyl halide in the reactant stream includes trifluoroacetyl iodide, the purified final product composition comprises less than 500 ppm (part per million by weight), less than 400 ppm, less than 300 ppm, less than 200 ppm, less than 100 ppm, or an amount within any two of the foregoing values of chlorotrifluoroethane. The final product composition may also comprise less than 500 ppm less than 400 ppm, less than 300 ppm, less than 200 ppm, less than 100 ppm, or an amount within any two of the foregoing values of hexafluoroethane. The final product composition may also comprise less than 500 ppm less than 400 ppm, less than 300 ppm, less than 200 ppm, less than 100 ppm, or an amount within any two of the foregoing values of trifluoromethane. The final product composition may also comprise less than 100 ppm, less than 90 ppm, less than 80 ppm, less than 70 ppm, less than 60 ppm, less than 50 ppm, less than 40 ppm, less than 30 ppm, less than 20 ppm, less than 10 ppm, or an amount within any of the two foregoing values of carbon monoxide. The final product composition may also comprise less than 1 ppm, less than 0.9 ppm, less than 0.8 ppm, less than 0.7 ppm, les than 0.6 ppm, less than 0.5 ppm, less than 0.4 ppm, less than 0.3 ppm, less than 0.2 ppm, less than 0.1 ppm, or an amount within any of the two foregoing values of hydrogen chloride. It is preferred that the purified final product stream comprises less than 250 ppm of chlorotrifluoroethane, for example less than 200 ppm, less than 150 ppm, less than 100 ppm, less than 50 ppm, or an amount within any of the two foregoing values of chlorotrifluoroethane. It is preferred that the purified final product comprises less than 250 ppm hexafluoroethane, for example less than 200 ppm, less than 150 ppm, less than 100 ppm, less than 50 ppm, or an amount within any of the two foregoing values of hexafluoroethane. It is preferred that the purified final product comprises less than 250 ppm trifluoromethane, for example, less than 200 ppm, less than 150 ppm, less than 100 ppm, less than 50 ppm, or an amount within any of the two foregoing values of trifluoromethane. It is preferred that the purified final product comprises less than 50 ppm carbon monoxide, for example, less than 40 ppm, less than 30 ppm, less than 20 ppm, less than 10 ppm, or an amount within any of the two foregoing values of carbon monoxide. It is preferred that the purified final product comprises less than 0.5 ppm hydrogen chloride, for example, less than 0.4 ppm, less than 0.3 ppm, less than 0.2 ppm, less than 0.1 ppm, or an amount within any of the two foregoing values of carbon monoxide. It is more preferred that the purified final product stream comprises less than 100 ppm of chlorotrifluoroethane, for example, less than 90 ppm, less than 80 ppm, less than 70 ppm, less than 60 ppm, less than 50 ppm, less than 40 ppm, less than 30 ppm, less than 20 ppm, less than 10 ppm, or an amount within any of the two foregoing values of chlorotrifluoroethane. It is more preferred that the purified final product stream comprises less than 10 ppm hexafluoroethane, for example, less than 9 ppm, less than 8 ppm, less than 7 ppm, less than 6 ppm, less than 5 ppm, less than 4 ppm, less than 3 ppm, less than 2 ppm, less than 1 ppm, or an amount within any of the two foregoing values of hexafluoroethane. It is more preferred that the purified final product stream comprises less than 100 ppm trifluoromethane, for example less than 90 ppm, less than 80 ppm, less than 70 ppm, less than 60 ppm, less than 50 ppm, less than 40 ppm, less than 30 ppm, less than 20 ppm, less than 10 ppm, or an amount within any of the two foregoing values of trifluoromethane. It is more preferred that the final product stream comprises less than 10 ppm carbon monoxide, for example, less than 9 ppm, less than 8 ppm, less than 7 ppm, less than 6 ppm, less than 5 ppm, less than 4 ppm, less than 3 ppm, less than 2 ppm, less than 1 ppm, or an amount within any of the two foregoing values of carbon monoxide. It is more preferred that the final product stream comprises less than 0.2 ppm hydrogen chloride, for example, less than 0.1 ppm, less than 0.09 ppm, less than 0.08 ppm, less than 0.07 ppm, less than 0.06 ppm, less than 0.05 ppm, less than 0.04 ppm, less than 0.03 ppm, less than 0.02 ppm, less than 0.01 ppm, or an amount within any of the two foregoing values of hydrogen chloride.

The purified final product composition may further comprise in amounts less than 500 ppm in total of compounds selected from the group consisting of trifluoroacetyl fluoride, hexafluoropropanone, trifluoroacetaldehyde, and trifluoroacetyl chloride. For example, the purified final product composition may comprise less than 400 ppm, less than 300 ppm, less than 200 ppm, less than 100 ppm, less than 50 ppm, or an amount within any of the two foregoing values of compounds selected from the group consisting of trifluoroacetyl fluoride, hexafluoropropanone, trifluoroacetaldehyde, and trifluoroacetyl chloride. It is preferred that the purified final product composition further comprises in amounts less than 250 ppm in total of compounds selected from the group consisting of trifluoroacetyl fluoride, hexafluoropropanone, trifluoroacetaldehyde, and trifluoroacetyl chloride. For example, the purified final product composition may comprise less than 200 ppm, less than 150 ppm, less than 100 ppm, less than 50 ppm, less than 25 ppm, or an amount within any of the two foregoing values of compounds selected from the group consisting of trifluoroacetyl fluoride, hexafluoropropanone, trifluoroacetaldehyde, and trifluoroacetyl chloride. It is more preferred that the purified final product composition further comprises in amounts less than 100 ppm in total of compounds selected from the group consisting of trifluoroacetyl fluoride, hexafluoropropanone, trifluoroacetaldehyde, and trifluoroacetyl chloride. For example, the purified final product composition may comprise less than 90 ppm, less than 80 ppm, less than 70 ppm, less than 60 ppm, less than 50 ppm, less than 40 ppm, less than 30 ppm, less than 20 ppm, less than 10 ppm, or an amount within any of the two foregoing values of compounds selected from the group consisting of trifluoroacetyl fluoride, hexafluoropropanone, trifluoroacetaldehyde, and trifluoroacetyl chloride.

Alternatively stated, if the trifluoroacetyl halide in the reactant stream includes trifluoroacetyl iodide, the purified final product composition may comprise at least 99 wt. % of trifluoroiodomethane, less than 500 ppm chlorotrifluoroethane, less than 500 ppm hexafluoroethane, less than 500 ppm trifluoromethane, less than 100 ppm carbon monoxide, less than 1 ppm hydrogen chloride and less than 500 ppm in total of compounds selected from the group consisting of trifluoroacetyl fluoride, hexafluoropropanone, trifluoroacetaldehyde, and trifluoroacetyl chloride. It is also provided that the purified final product composition may comprise at least 99.5 wt. % of trifluoroiodomethane, less than 250 ppm chlorotrifluoroethane, less than 250 ppm hexafluoroethane, less than 250 ppm trifluoromethane, less than 50 ppm carbon monoxide, less than 0.5 ppm hydrogen chloride and less than 250 ppm in total of compounds selected from the group consisting of trifluoroacetyl fluoride, hexafluoropropanone, trifluoroacetaldehyde, and trifluoroacetyl chloride. It is also provided that the purified final product composition may comprise at least 99.7 wt. % of trifluoroiodomethane, less than 100 ppm chlorotrifluoroethane, less than 100 ppm hexafluoroethane, less than 100 ppm trifluoromethane, less than 20 ppm carbon monoxide, less than 0.2 ppm hydrogen chloride and less than 100 ppm in total of compounds selected from the group consisting of trifluoroacetyl fluoride, hexafluoropropanone, trifluoroacetaldehyde, and trifluoroacetyl chloride. It is also provided that the purified final product composition may comprise at least 99.9 wt. % of trifluoroiodomethane, less than 100 ppm chlorotrifluoroethane, less than 100 ppm hexafluoroethane, less than 100 ppm trifluoromethane, less than 20 ppm carbon monoxide, less than 0.2 ppm hydrogen chloride and less than 100 ppm in total of compounds selected from the group consisting of trifluoroacetyl fluoride, hexafluoropropanone, trifluoroacetaldehyde, and trifluoroacetyl chloride.

Alternatively stated, if the trifluoroacetyl halide in the reactant stream includes trifluoroacetyl chloride, the purified final product composition may consist essentially of at least 99 wt. % of trifluoroiodomethane, less than 500 ppm chlorotrifluoroethane, less than 500 ppm hexafluoroethane, less than 500 ppm trifluoromethane, less than 100 ppm carbon monoxide, less than 1 ppm hydrogen chloride and the balance of compounds selected from the group consisting of trifluoroacetyl fluoride, hexafluoropropanone, trifluoroacetaldehyde, and trifluoroacetyl chloride. It is also provided that the purified final product composition may consist essentially of at least 99.5 wt. % of trifluoroiodomethane, less than 250 ppm chlorotrifluoroethane, less than 250 ppm hexafluoroethane, less than 250 ppm trifluoromethane, less than 50 ppm carbon monoxide, less than 0.5 ppm hydrogen chloride and the balance of compounds selected from the group consisting of trifluoroacetyl fluoride, hexafluoropropanone, trifluoroacetaldehyde, and trifluoroacetyl chloride. It is also provided that the purified final product composition may consist essentially of at least 99.7 wt. % of trifluoroiodomethane, less than 100 ppm chlorotrifluoroethane, less than 100 ppm hexafluoroethane, less than 100 ppm trifluoromethane, less than 20 ppm carbon monoxide, less than 0.2 ppm hydrogen chloride and the balance of compounds selected from the group consisting of trifluoroacetyl fluoride, hexafluoropropanone, trifluoroacetaldehyde, and trifluoroacetyl chloride. It is also provided that the purified final product composition may consist essentially of at least 99.9 wt. % of trifluoroiodomethane, less than 100 ppm chlorotrifluoroethane, less than 100 ppm hexafluoroethane, less than 100 ppm trifluoromethane, less than 100 ppm carbon monoxide, less than 0.2 ppm hydrogen chloride and the balance of compounds selected from the group consisting of trifluoroacetyl fluoride, hexafluoropropanone, trifluoroacetaldehyde, and trifluoroacetyl chloride.

Alternatively stated, if the trifluoroacetyl halide in the reactant stream includes trifluoroacetyl chloride, the purified final product composition may consist of at least 99 wt. % of trifluoroiodomethane, less than 500 ppm chlorotrifluoroethane, less than 500 ppm hexafluoroethane, less than 500 ppm trifluoromethane, less than 100 ppm carbon monoxide, less than 1 ppm hydrogen chloride and the balance of compounds selected from the group consisting of trifluoroacetyl fluoride, hexafluoropropanone, trifluoroacetaldehyde, and trifluoroacetyl chloride. It is also provided that the purified final product composition may consist of at least 99.5 wt. % of trifluoroiodomethane, less than 250 ppm chlorotrifluoroethane, less than 250 ppm hexafluoroethane, less than 250 ppm trifluoromethane, less than 50 ppm carbon monoxide, less than 0.5 ppm hydrogen chloride and the balance of compounds selected from the group consisting of trifluoroacetyl fluoride, hexafluoropropanone, trifluoroacetaldehyde, and trifluoroacetyl chloride. It is also provided that the purified final product composition may consist of at least 99.7 wt. % of trifluoroiodomethane, less than 100 ppm chlorotrifluoroethane, less than 100 ppm hexafluoroethane, less than 100 ppm trifluoromethane, less than 20 ppm carbon monoxide, less than 0.2 ppm hydrogen chloride and the balance of compounds selected from the group consisting of trifluoroacetyl fluoride, hexafluoropropanone, trifluoroacetaldehyde, and trifluoroacetyl chloride. It is also provided that the purified final product composition may consist of at least 99.9 wt. % of trifluoroiodomethane, less than 100 ppm chlorotrifluoroethane, less than 100 ppm hexafluoroethane, less than 100 ppm trifluoromethane, less than 100 ppm carbon monoxide, less than 0.2 ppm hydrogen chloride and the balance of compounds selected from the group consisting of trifluoroacetyl fluoride, hexafluoropropanone, trifluoroacetaldehyde, and trifluoroacetyl chloride.

The amount of methyl propane in the CF₃I product may be about 150 ppm or less, about 100 ppm or less, about 90 or less, about 80 ppm or less, about 70 ppm or less, about 60 ppm or less, about 50 ppm or less, about 40 ppm or less, about 30 ppm or less, about 20 ppm or less, about 10 ppm or less or within any range defined between any of the two foregoing values, as measured by GC FID area %.

It has been found that the purified final product stream of the three-step gas-phase process described above results in a high-purity trifluoroiodomethane product due to the high purity of the trifluoroacetyl iodide in the purified intermediate product stream. The three-step gas-phase process produces surprisingly good process yields and is amenable for the manufacture of trifluoroiodomethane on a commercial scale.

While this invention has been described as relative to exemplary designs, the present invention may be further modified within the spirit and scope of this disclosure. Further, this application is intended to cover such departures from the present disclosure as come within known or customary practice in the art to which this invention pertains.

As used herein, the phrase “within any range defined between any two of the foregoing values” literally means that any range may be selected from any two of the values listed prior to such phrase regardless of whether the values are in the lower part of the listing or in the higher part of the listing. For example, a pair of values may be selected from two lower values, two higher values, or a lower value and a higher value.

### Examples

### Example 1: Reaction of TFAC with Purified HI

Activated carbon catalyst (Norit ROX0.8, 74 mL) is loaded into an ¾″ Inconel 600 reactor. The reactor is preheated to 90° C. with the reactor outlet pressure controlled at 70 psig. 120 g/h of TFAC (containing 99.84% TFAC, 0.05% 133a and 0.11% TFA by GC area %) and 82.8 g/h of HI (Purified HI with iodo-methane and iodo-propane removed) are fed into the reactor, which give a TFAC/HI molar ratio of 1.40 and a contact time of 33.3 seconds. The average TFAC conversion of 70 mol % is observed with no methyl propane detected in the reactor effluent samples.

### Comparative Example A: Reaction of TFAC with Crude HI

Activated carbon catalyst (Norit ROX0.8, 74 mL) was loaded into an ¾″ Inconel 600 reactor. The reactor was preheated to 90° C. with the reactor outlet pressure controlled at 70 psig. 120 g/h of TFAC (containing 99.84% TFAC, 0.05% 133a and 0.11% TFA by GC area %) and 82.8 g/h of HI (HI made in house, crude without purification, contained iodo-methane and iodo-propane) were fed into the reactor, which gave a TFAC/HI molar ratio of 1.40 and a contact time of 33.3 seconds. During 188 hours of continuous reaction, the average TFAC conversion of 69.2 mol % was observed with an average methyl propane concentration of 51 ppm in the reactor effluent samples.

### Example 2: Purification of TFAI

148.5 lbs of TFAI crude material was charged to a batch distillation column. The crude material contained about 91.8 GC area % TFAI and 8.2 GC area % impurities. The concentration of the undesired methyl propane impurity was determined to be 0.0174 GC area % by GC/MS flame ionization detection (FID) analysis. The ratio of TFAI to methyl propane was 5279.4:1.

The batch distillation column consisted of a 10 gallon reboiler, shell and tube condenser, and a 2″ inner diameter by 10′ tall column packed with high efficiency Pro-Pak® random column packing. The column had about 35 theoretical plates. The distillation column was equipped with temperature, pressure and differential pressure transmitters, and distillate take-off flow control.

About 3.9 lbs of a lights cut was recovered from the overhead of the column in a dry ice trap, which consisted mainly of CF₃I, but also contained 1.0047 GC area % TFAI by GC analysis while the concentration of methyl propane was analyzed to be 0.3897 GC area %. The ratio of TFAI to methyl propane was 2.58:1 showing that methyl propane was indeed enriched in the overhead stream of the column and it could be separated from TFAI by conventional distillation. During the lights cut, the distillation column pressure ranged from 30 psig-5 psig, the pressure delta across the column ranged from 25-30″ H₂O, and the column overhead temperature ranged from 0° C.-20° C. (pressure and composition dependent).

Following the removal of lights, the main distillate cut was started. The distillation was considered completed once there was not sufficient material in the reboiler to maintain reflux. Upon the completion of distillation, about 138.5 lbs of main distillate cut was collected into a product collection cylinder and analyzed by GC/MS FID. During the main cut, the distillation column pressure ranged from 5-3 psig, the pressure delta across the column ranged from 25-30″ H₂O, and the column overhead temperature ranged from 38-28° C. (pressure dependent).

The GC/MS FID analysis showed 0.0040 FID area % of methyl propane and 96.51 FID area % of TFAI. The methyl propane concentration in main distillate cut was much lower than that in TFAI crude once again showing that conventional distillation did reduce the amount of the undesired impurity.

Table 1 below shows the full GC/MS FID analysis of the TFAI crude and lights cut.

TABLE 1

| | Area % FID | |
|---|---|---|
| Component | TFAI Crude | Lights Cut |
| Trifluoromethane (CF₃H) | 0.0248 | 0.2161 |
| Hexafluoropropene (FO-1216) | 0.0237 | 0.1718 |
| Trifluoroacetyl chloride (TFAC) | 0.4965 | 1.7244 |
| Trifluoroacetic acid anhydride (TFAA) | 0.2378 | 0.8223 |
| Idotrifluoromethane (CF₃I) | 5.8795 | 92.8111 |
| Methyl propane | 0.0174 | 0.3897 |
| 2-Chloro-1,1,1-trifluoroethane (HCFC-133a) | 0.0244 | 1.5357 |
| Iodopentafluoroethane (C₂F₅I) | 0.0277 | 1.1559 |
| E-isomer of 1-chloro-3,3,3-trifluoropropene | 0.0070 | 0.0629 |
| (HCFO-1233zdE) | | |
| Hexafluoroacetone (CF₃C(O)CF₃) | 0.0046 | 0.0739 |
| Trifluoroacetyl iodide (TFAI) | 91.8548 | 1.0047 |
| Iodomethane (CH₃I) | 0.0176 | 0.0314 |
| Iodopropane (C₃H₇I) | 0.0306 | Not detected |
| Trifluoroacetic acid (TFA) | 1.2367 | Not detected |
| Iodopropene (C₃H₅I) | 0.0048 | Not detected |
| Di-iodopropane (C₃H₆I₂) | 0.0631 | Not detected |
| Others¹ | 0.0489 | Not detected |

¹Others include octafluorobutanone, Z-isomer of 1-chloro-3,3,3-trifluoropropene (HCFO-1233zdZ), methyl pentane isomer, methyl cyclopentane, and unknowns.

### Example 3: Purification of CF₃I

A batch distillation column consisting of a 10 gallon reboiler, shell and tube condenser, and a 2″ inner diameter by 10′ tall column packed with high efficiency Pro-Pak® random column packing was used to purify crude CF₃I. The column had about 35 theoretical plates. The distillation column was equipped with temperature, pressure and differential pressure transmitters, and distillate take-off flow control.

153 lbs of CF₃I crude was charged to the reboiler. The GC analysis of the crude showed that the purity of the CF₃I (nBP=−22.5° C.) was 99.874 GC area % and the concentration of methyl propane (nBP=−11.7° C.) was 0.0248 GC area %. There were also various other lower and higher boiling impurities present in the crude.

First the non-condensibles and lights were removed off the top of the condenser and the purity of the stream was monitored by GC analysis. When the low boiling impurities (mostly CHF₃) were non-detectable, the main cut was started by directing the distillate stream to a product collection cylinder at a rate of 0.9-1.1 lb/hr. During the main cut, the distillation column pressure ranged from 43.5-51 psig, the pressure delta across the column ranged from 23-26″ H₂O, and the column overhead temperature ranged from 17.5-21.5° C. (pressure dependent). The purity of the distillate was greater than 99.99% by GC, with no light impurities and only a single high-boiling impurity (methyl propane). The amount of methyl propane in the distillate samples started at 0.0030 GC area % and as the distillation progressed, it slowly increased to 0.0240 GC area % (0.0030→0.0240 GC area %). The distillation was considered completed when there was no longer sufficient material in the reboiler to maintain reflux.

In total 137 lbs of distillate was collected in the distillation. The CF₃I that was collected has a purity of 99.9937 GC Area % with methyl propane as the only impurity at 0.0063 GC Area %. This data demonstrates the difficulty in completely removing this impurity from CF₃I by conventional distillation.

The reboiler was drained and 8.3 lbs of reboiler residue were collected for which a GC analysis can be found in Table 2 below, along with results of the GC analysis of the crude CF₃I and the main cut.

TABLE 2

| | | Composition, GC area % | | | |
|---|---|---|---|---|---|
| | Weight, | | | Methyl | |
| Material | lb | CHF₃ | CF₃I | propane | Others¹ |
| CF₃I crude | 153.0 | 0.0857 | 99.8740 | 0.0248 | 0.1012 |
| Main cut | 137.0 | Not detected | 99.9937 | 0.0063 | Not detected |
| Reboiler | 8.3 | Not detected | 99.4624 | 0.2460 | 0.2916 |
| residue | | | | | |

¹Others include HCFC-133a, C₂F₅I, CH₃I, etc.

### Example 4: Purification of CF₃I

A second batch distillation using a different batch of crude CF₃I is performed with the same equipment and conditions as described above. 140 lbs of CF₃I crude is charged to the system. The GC analysis of the crude shows that the purity of the CF₃I is 99.680 GC area % and the concentration of methyl propane is 0.0142 GC area %. There are also various other lower and higher boiling impurities present in the crude CF₃I.

First the non-condensibles and lights are removed off the top of the condenser and the purity of the stream is monitored by GC analysis. When the low boiling impurities such as trifluoromethane (CHF₃) are non-detectable, the main cut is started by directing the distillate stream to a product collection cylinder at a rate of 0.9-1.1 lb/hr. The purity of the distillate is greater than 99.99 GC area %, with no light impurities and only a single high-boiling impurity, methyl propane. The distillation is considered complete when there is not sufficient material remaining in the reboiler to maintain adequate reflux. In total, 124.7 lbs of distillate are collected in the distillation.

The concentration of CF₃I in the purified product is 99.9962 GC Area % with methyl propane as the only impurity at 38 ppm GC Area %. This data demonstrates both the difficulty in completely removing this impurity from CF₃I by conventional distillation, and that reducing the concentration of methyl propane in the crude CF₃I by first purifying the TFAI feedstock used to produce it can ultimately produce CF₃I with lower concentrations of methyl propane impurities.

The reboiler is drained and 6.1 lbs of reboiler residue are collected for GC analysis. The results are shown below in Table 3, along with the results of analysis of the crude CF₃I and the main cut.

TABLE 3

| | | Composition, GC area % | | | |
|---|---|---|---|---|---|
| | Weight, | | | Methyl | |
| Material | lb | CHF₃ | CF₃I | propane | Others¹ |
| CF₃I crude | 140 | 0.1994 | 99.6796 | 0.0142 | 0.1068 |
| Main cut | 124.7 | Not detected | 99.9962 | 0.0038 | Not detected |
| Reboiler | 6.1 | Not detected | 99.4624 | 0.1389 | 0.3029 |
| residue | | | | | |

¹Others include HCFC-133a, C₂F₅I, CH₃I, etc.

### Example 5: Purification of TFAI by Extraction

This example illustrates the removal of methyl propane from a mixture of methyl propane and Trifluoroacetyl iodide (TFAI) according to certain preferred embodiments of the present invention.

A Trifluoroacetyl iodide (TFAI) crude stream which contains about 0.0750 GC area % methyl propane is vaporized and fed to the bottom of a packed column at a feed rate of about 2.4 lbs per hour for about 4 hours. A liquid stream of n-octane is fed continuously to the top of the same packed column at a feed rate of about 3.6 lbs per hour during the same time frame. A gaseous stream exiting the top of the column comprises TFAI with less than 0.090 GC area % methyl propane therein. The concentration of methyl propane in the n-octane increases from non-detectable to about 0.044%

### Example 6: Purification of TFAI by Adsorption on 13× Molecular Sieves

This example illustrates the removal of methyl propane from a mixture of methyl propane and Trifluoroacetyl iodide (TFAI) according to certain preferred embodiments of the present invention.

A liquid Trifluoroacetyl iodide (TFAI) crude stream which contains about 0.0750 GC area % methyl propane is fed to the bottom of a 1″ ID×36″ L column filled with 450 cc of molecular sieve 13× adsorbent at a feed rate of about 0.25 lbs per hour for about 10 hours. The material exiting the top of the column is collected in a cylinder. 8.8 lbs total is collected in the cylinder and is analyzed by gas chromatography. The amount of methyl propane in the TFAI has been reduced to 0.0075 GC area %.

### Example 7: Purification of TFAI by Adsorption on Activated Carbon

This example illustrates the removal of methyl propane from a mixture of methyl propane and Trifluoroacetyl iodide (TFAI) according to certain preferred embodiments of the present invention.

A liquid Trifluoroacetyl iodide (TFAI) crude stream which contains about 0.0750 GC area % methyl propane is fed to the bottom of a 1″ IDλ36″ L column filled with 450 cc of BAX 1500 activated carbon at a feed rate of about 0.25 lbs per hour for about 10 hours. The material exiting the top of the column is collected in a cylinder. 8.7 lbs total is collected in the cylinder and is analyzed by gas chromatography. The amount of methyl propane in the TFAI has been reduced to 0.0071 GC area %.

### Example 8: Purification of TFAI by Adsorption on Zeolite ZSM-5

This example illustrates the removal of methyl propane from a mixture of methyl propane and Trifluoroacetyl iodide (TFAI) according to certain preferred embodiments of the present invention.

A liquid Trifluoroacetyl iodide (TFAI) crude stream which contains about 0.0750 GC area % methyl propane is fed to the bottom of a 1″ ID×36″ L column filled with 450 cc of Zeolite ZSM-5 at a feed rate of about 0.25 lbs per hour for about 10 hours. The material exiting the top of the column is collected in a cylinder. 8.9 lbs total is collected in the cylinder and is analyzed by gas chromatography. The amount of methyl propane in the TFAI has been reduced to 0.0068 GC area %.

### Example 9: Purification of TFAI by Extraction

This example illustrates the removal of methyl propane from a mixture of methyl propane and trifluoroiodomethane (CF₃I) according to certain preferred embodiments of the present invention.

A trifluoroiodomethane (CF₃I) crude stream which contains about 0.0500 GC area % methyl propane is vaporized and fed to the bottom of a packed column at a feed rate of about 2.6 lbs per hour for about 4 hours. A liquid stream of chlorobenzene is fed continuously to the top of the same packed column at a feed rate of about 3.5 lbs per hour during the same time frame. A gaseous stream exiting the top of the column comprises CF₃I with less than 0.009 GC area % methyl propane therein. The concentration of methyl propane in the chlorobenzene increases from non-detectable to about 0.036%

### Examples 10-18: Purification of CF₃I by Adsorbents

These examples illustrate the removal of methyl propane from a mixture of methyl propane and trifluoroiodomethane according to certain preferred embodiments of the present invention.

A total of 9 experiments are conducted to study the removal of methyl propane from a crude trifluoroiodomethane (CF₃I) stream using solid adsorbents. The procedure used for the study is as follows: A CF₃I crude stream which contains 0.0307 GC area % methyl propane is vaporized and fed to the top of a 1″ ID×36″ L column filled with 450 cc of a solid adsorbent chosen from molecular sieves, activated carbon, carbon molecular sieves, and activated alumina. The CF₃I is fed continuously through the column at a feed rate of about 0.25 lbs per hour for about 10 hours. The material exiting the bottom of the column is collected into an evacuated cylinder that is cooled using dry ice. The composite of the liquid CF₃I that was collected is analyzed by gas chromatography. The amount of methyl propane in the CF₃I is reduced to varying degrees by all the solid adsorbents tested.

TABLE 4

| | | | CF₃I crude | methyl propane | methyl propane | methyl propane |
|---|---|---|---|---|---|---|
| Experiment | | volume | feed rate | concentration | concentration | concentration |
| no. | Adsorbent | (cc) | (g/hr) | IN (GC area %) | OUT (GC area %) | reduction (%) |
| 1 | 3Å mol sieves | 450 | 113 | 0.0307 | <0.0200 | 34.9 |
| 2 | 4Å mol sieves | 450 | 113 | 0.0307 | <0.0200 | 34.9 |
| 3 | XH-9 molsieves | 450 | 113 | 0.0307 | <0.0100 | 67.4 |
| 4 | 13X mol sieves | 450 | 113 | 0.0307 | <0.0200 | 93.5 |
| 5 | MSC-3K 172 carbon | 450 | 113 | 0.0307 | <0.0100 | 67.4 |
| | molecular sieves | | | | | |
| 6 | SAS40 1/8″ Alumina | 450 | 113 | 0.0307 | <0.0200 | 34.9 |
| 7 | CBV5524G CY zeolite | 450 | 113 | 0.0307 | <0.0100 | 67.4 |
| | ammonium powder | | | | | |
| 8 | BAX 1500 activated | 450 | 113 | 0.0307 | <0.0020 | 93.5 |
| | carbon | | | | | |
| 9 | ZSM-S mol sieves | 450 | 113 | 0.0307 | <0,0020 | 93.5 |

### Aspects

Aspect 1 is a process for producing trifluoroiodomethane (CF₃I) with a low concentration of methyl propane, the process comprising: providing a first reactant stream comprising hydrogen iodide (HI); reacting the first reactant stream with a second reactant stream comprising trifluoroacetyl chloride (TFAC) to produce an intermediate product stream comprising trifluoroacetyl iodide (TFAI); purifying the intermediate product stream to remove methyl propane; and reacting the purified intermediate product stream to produce a final product stream comprising trifluoroiodomethane (CF₃I).

Aspect 2 is the process of Aspect 1, wherein the amount of iodomethane in the hydrogen iodide (HI) is less than 250 ppm.

Aspect 3 is the process of Aspect 1 or Aspect 2, wherein the amount of iodopropane in the hydrogen iodide (HI) is less than 250 ppm.

Aspect 4 is the process of any of Aspects 1 to 3, wherein the trifluoroacetyl iodide (TFAI) is purified by distillation.

Aspect 5 is the process of any one of Aspects 1 to 4, wherein the trifluoroacetyl iodide (TFAI) is purified by solvent extraction.

Aspect 6 is the process of Aspect 5, wherein the solvent used for the solvent extraction is selected from the group consisting of hydrocarbons and chlorinated compounds.

Aspect 7 is the process of Aspect 5 or Aspect 6, wherein the solvent used for the solvent extraction is regenerated by distillation.

Aspect 8 is the process of any one of Aspects 1 to 3, wherein the trifluoroacetyl iodide (TFAI) is purified with an adsorbent.

Aspect 9 is the process of Aspect 8, wherein the adsorbent is selected from the group consisting of molecular sieves, carbon, carbon molecular sieves, alumina, and zeolites.

Aspect 10 is the process of any one of Aspects 1 to 9, wherein the concentration of trifluoroacetyl iodide (TFAI) in the purified intermediate product stream is greater than about 99 wt. % based on the total weight of the composition.

Aspect 11 is the process of any one of Aspects 1 to 10, wherein the amount of methyl propane in the in purified intermediate product stream is about 50 ppm or less.

Aspect 12 is the process of any one of Aspects 1 to 11, further comprising purifying the final product stream comprising trifluoroiodomethane (CF₃I) by distillation.

Aspect 13 is the process of any one of Aspects 1 to 12, further comprising purifying the final product stream comprising trifluoroiodomethane (CF₃I) with an adsorbent.

Aspect 14 is the process of Aspect 13, wherein the adsorbent is selected from the group consisting of molecular sieves, carbon, carbon molecular sieves, alumina, and zeolites.

Aspect 15 is the process of any one of Aspects 1 to 14, wherein the amount of methyl propane in the trifluoroiodomethane (CF₃I) product is about 70 ppm or less.

Aspect 16 is a composition comprising: at least 99 wt. % of trifluoroiodomethane; less than 500 ppm chlorotrifluoroethane; less than 500 ppm hexafluoroethane; less than 500 ppm trifluoromethane; less than 100 ppm carbon monoxide; less than 100 ppm carbon methyl propane and less than 1 ppm hydrogen chloride.

Aspect 17 is the composition of Aspect 16, further comprising: from 1 ppm to 500 ppm in total of compounds selected from the group consisting of trifluoroacetyl fluoride, hexafluoropropanone, trifluoroacetaldehyde, and trifluoroacetyl chloride.

Aspect 18 is the composition of Aspect 16, wherein the amount of methyl propane less than 70 ppm.

Aspect 19 is a process for producing trifluoroacetyl iodide (TFAI) with a low concentration of methyl propane, the process comprising: providing a first reactant stream comprising a purified stream of hydrogen iodide (HI); reacting the first reactant stream with a second reactant stream comprising trifluoroacetyl chloride (TFAC) to produce a final product stream comprising trifluoroacetyl iodide (TFAI).

Aspect 20 is the process of Aspect 19, wherein the amount of iodomethane in the hydrogen iodide (HI) is less than 250 ppm.

Aspect 21 is the process of Aspect 19 or Aspect 20, wherein the amount of iodopropane in the hydrogen iodide (HI) is less than 250 ppm.

What is claimed is:

1. A process for producing trifluoroiodomethane (CF₃I) with a low concentration of methyl propane, the process comprising:
providing a first reactant stream comprising hydrogen iodide (HI);
reacting the first reactant stream with a second reactant stream comprising trifluoroacetyl chloride (TFAC) to produce an intermediate product stream comprising trifluoroacetyl iodide (TFAI);
purifying the intermediate product stream to remove methyl propane; and
reacting the purified intermediate product stream to produce a final product stream comprising trifluoroiodomethane (CF₃I).

2. The process of claim 1, wherein the amount of iodomethane in the hydrogen iodide (HI) is less than 250 ppm.

3. The process of claim 1, wherein the amount of iodopropane in the hydrogen iodide (HI) is less than 250 ppm.

4. The process of claim 1, wherein the trifluoroacetyl iodide (TFAI) is purified by distillation.

5. The process of claim 1, wherein the trifluoroacetyl iodide (TFAI) is purified by solvent extraction.

6. The process of claim 5, wherein the solvent used for the solvent extraction is selected from the group consisting of hydrocarbons and chlorinated compounds.

7. The process of claim 5, wherein the solvent used for the solvent extraction is regenerated by distillation.

8. The process of claim 1, wherein the trifluoroacetyl iodide (TFAI) is purified with an adsorbent.

9. The process of claim 8, wherein the adsorbent is selected from the group consisting of molecular sieves, carbon, carbon molecular sieves, alumina, and zeolites.

10. The process of claim 1, wherein the concentration of trifluoroacetyl iodide (TFAI) in the purified intermediate product stream is greater than about 99 wt. % based on the total weight of the composition.

11. The process of claim 1, wherein the amount of methyl propane in the in purified intermediate product stream is about 50 ppm or less.

12. The process of claim 1, further comprising purifying the final product stream comprising trifluoroiodomethane (CF₃I) by distillation.

13. The process of claim 1, further comprising purifying the final product stream comprising trifluoroiodomethane (CF₃I) with an adsorbent.

14. The process of claim 13, wherein the adsorbent is selected from the group consisting of molecular sieves, carbon, carbon molecular sieves, alumina, and zeolites.

15. The process of claim 1, wherein the amount of methyl propane in the trifluoroiodomethane (CF₃I) product is about 70 ppm or less.

16. A composition comprising:
at least 99 wt. % of trifluoroiodomethane
less than 500 ppm chlorotrifluoroethane;
less than 500 ppm hexafluoroethane;
less than 500 ppm trifluoromethane;
less than 100 ppm carbon monoxide;
less than 100 ppm carbon methyl propane and
less than 1 ppm hydrogen chloride.

17. The composition of claim 16, further comprising:
from 1 ppm to 500 ppm in total of compounds selected from the group consisting of trifluoroacetyl fluoride, hexafluoropropanone, trifluoroacetaldehyde, and trifluoroacetyl chloride.

18. The composition of claim 16, wherein the amount of methyl propane less than 70 ppm.

19. A process for producing trifluoroacetyl iodide (TFAI) with a low concentration of methyl propane, the process comprising:
providing a first reactant stream comprising a purified stream of hydrogen iodide (HI);
reacting the first reactant stream with a second reactant stream comprising trifluoroacetyl chloride (TFAC) to produce a final product stream comprising trifluoroacetyl iodide (TFAI).

20. The process of claim 19, wherein the amount of iodomethane in the hydrogen iodide (HI) is less than 250 ppm.
